(19) United States
(12) Patent Application Publication
(10) Pub. No.: US 2009/0270395 A1
(43) Pub. Date: Oct. 29, 2009

(54) EP4 Receptor Agonist, Compositions and Methods Thereof

(76) Inventors: John Colucci, Kirkland (CA); Yongxin Han, Kirkland (CA); Julie A. Farand, Montreal (CA)

Correspondence Address: MERCK AND CO., INC, P O BOX 2000, RAHWAY, NJ, 07065-0907, US

(21) Appl. No.: 11/989,747

(22) PCT Filed: Jul. 28, 2006
(86) PCT No.: PCT/CA2006/001254

§ 371 Date: Jan. 28, 2008

Related U.S. Application Data
(60) Provisional application No. 60/705,120, filed on Aug. 3, 2005.

(51) Int. Cl. A61K 31/5377; C07D 265/06; A61K 31/535; A61P 27/02; A61P 19/00

(52) U.S. Cl. 514/236.2; 544/97; 514/228.8

(57) ABSTRACT

This invention relates to potent selective agonists of the EP₄ subtype of prostaglandin E₂ receptors, their use or a formulation thereof in the treatment of glaucoma and other conditions, which are related to elevated intraocular pressure in the eye of a patient. This invention further relates to the use of the compounds of this invention for mediating the bone modeling and remodeling processes of the osteoblasts and osteoclasts. The compounds of the present invention are the compounds of Formula (I).

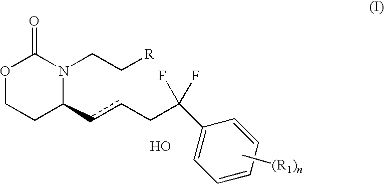

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

Many of the drugs formerly used to treat glaucoma proved unsatisfactory. Current methods of treating glaucoma include using therapeutic agents such as pilocarpine, carbonic anhydrase inhibitors, beta-blockers, prostaglandins and the like. However, these therapies often produce undesirable local effects. As can be seen, there are several current therapies for treating glaucoma and elevated intraocular pressure, but the efficacy and the side effect profiles of these agents are not ideal. Therefore, there still exists the need for new and effective therapies with little or no side effects.

A variety of disorders in humans and other mammals involve or are associated with abnormal or excessive bone loss. Such disorders include, but are not limited to, osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Prostaglandins such as the PGE₂ series are known to stimulate bone formation and increase bone mass in mammals, including man. It is believed that the four different receptor subtypes, designated EP₁, EP₂, EP₃, and EP₄ are involved in mediating the bone modeling and remodeling processes of the osteoblasts and osteoclasts. The major prostaglandin receptor in bone is EP₄, which is believed to provide its effect by signaling via cyclic AMP. In the present invention it is found that the formula I agonists of the EP₄ subtype receptor are useful for stimulating bone formation. WO 02/24647, WO 02/42268, EP 1114816, WO 01/46140 and WO 01/72268 disclose EP₄ agonists. However, they do not disclose the compounds of the instant invention.

### SUMMARY OF THE INVENTION

This invention relates to agonists of the EP₄ subtype of prostaglandin E2 receptors and their use or a formulation thereof in the treatment of glaucoma and other conditions that are related to elevated intraocular pressure in the eye of a patient. In particular, this invention relates to a series of 1,3-oxazinan-2-one, and 4,5-disubstituted morpholin-3-one derivatives and their use to treat ocular diseases and to provide a neuroprotective effect to the eye of mammalian species, particularly humans. This invention further relates to the use of the compounds of this invention for mediating the bone modeling and remodeling processes of the osteoblasts and osteoclasts.

More particularly, this invention relates to novel EP₄ agonist having the structural formula I:

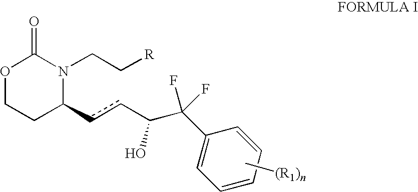

or a pharmaceutically acceptable salt, enantiomer, diastereomer, prodrug or mixture thereof, wherein,
R represents (CH₂)ₓCOOR₃, (CH₂)ₙC₃₋₁₀ cycloalkyl; —(CH₂)ₙC₃₋₁₀ heterocyclyl, (CH₂)ₙC₆₋₁₀ aryl, said cycloalkyl, heterocyclyl, and aryl substituted with R₂; provided that when R is —(CH₂)ₙC₃₋₁₀ heterocyclyl it does not represent thienyl;
R₁ independently represents hydrogen, C₁₋₆ alkyl, halogen, CF₃, aryl, said aryl optionally substituted with 1 to 3 groups of halogen, C₁₋₆ alkyl, CF₃, or N(R₄)₂
R₂ represents COOR₃ or a carboxylic acid isostere;
R₃ and R₄ independently represent H, or C₁₋₆ alkyl;
n represents 0-3;
x represents 2-5; and
--- represents a double or single bond.

This and other aspects of the invention will be realized upon inspection of the invention as a whole.

### DETAILED DESCRIPTION OF THE INVENTION

The invention is described herein in detail using the terms defined below unless otherwise specified.

The term “therapeutically effective amount”, as used herein, means that amount of the EP₄ receptor subtype agonist of formula I, or other actives of the present invention, that will elicit the desired therapeutic effect or response or provide the desired benefit when administered in accordance with the desired treatment regimen. A preferred therapeutically effective amount relating to the treatment of abnormal bone resorption is a bone formation, stimulating amount. Likewise, a preferred therapeutically effective amount relating to the treatment of ocular hypertension or glaucoma is an amount effective for reducing intraocular pressure and/or treating ocular hypertension and/or glaucoma.

“Pharmaceutically acceptable” as used herein, means generally suitable for administration to a mammal, including humans, from a toxicity or safety standpoint.

The term “prodrug” refers to compounds which are drug precursors which, following administration and absorption, release the claimed drug in vivo via some metabolic process. A non-limiting example of a prodrug of the compounds of this invention would be an ester of an acid group, where the ester is easily hydrolyzed to the active acid after administration to a patient. Exemplary prodrugs include acetic acid derivatives that are non-narcotic, analgesics/non-steroidal, anti-inflammatory drugs having a free CH₂COOH group (which can optionally be in the form of a pharmaceutically acceptable salt, e.g. —CH₂COO—Na+), typically attached to a ring system, preferably to an aromatic or heteroaromatic ring system.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190)

When any variable (e.g. aryl, heterocycle, R₁, etc.) occurs more than one time in any constituent, its definition on each occurrence is independent at every other occurrence. Also, combinations of substituents/or variables are permissible only if such combinations result in stable compounds.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopropyl cyclopentyl and cyclohexyl. When the alkyl group is said to be substituted with an alkyl group, this is used interchangeably with “branched alkyl group”.

Cycloalkyl is a species of alkyl containing from 3 to 10 carbon atoms, unless otherwise defined, without alternating or resonating double bonds between carbon atoms. It may contain from 1 to 3 rings, which are fused. Examples of such cycloalkyl elements include, but are not limited to, cyclopropyl, cyclobutyl, cyclopentyl, cyclohexyl and cycloheptyl.

Halogen (halo) refers to chlorine, fluorine, iodine or bromine.

Carboxylic isostere represents tetrazole, acylsulfonamide, sulfonic acid, phosphonic acid or prodrug such as C₁₋₆ aldehyde or C₁₋₆ alcohol.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to two such rings being present, containing up to 10 atoms therein, with alternating (resonating) double bonds between adjacent carbon atoms or suitable heteroatoms. Examples of aryl groups are phenyl, naphthyl, tetrahydronaphthyl, indanyl, and biphenyl, preferably phenyl, naphthyl or biphenyl. Aryl groups may likewise be substituted as defined. Preferred substituted aryls include phenyl and naphthyl.

The term heterocyclyl or heterocyclic, as used herein, represents a stable 3- to 7-membered monocyclic or stable 8- to 10-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, dihydropyrrolyl, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. Preferably, heterocycle is selected from 2-azepinonyl, benzimidazolyl, 2-diazapinonyl, dihydroimidazolyl, dihydropyrrolyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, and tetrahydroisoquinolinyl.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl and triazolyl. Additional nitrogen atoms may be present together with the first nitrogen and oxygen or sulfur, giving, e.g., thiadiazole.

The term “agonist” as used herein means EP₄ subtype compounds of formula I interact with the EP4 receptor to produce maximal, super maximal or submaximal effects compared to the natural agonist, PGE2. See Goodman and Gilman, The Pharmacological Basis of Therapeutics, 9^{th} edition, 1996, chapter 2.

One embodiment of this invention is realized when R is (CH₂)ₓCOOR₃ and all other variables are as originally defined. A subembodiment of this invention is realized when x is 3-4. Another subembodiment is realized when R₃ is H. Still another subembodiment is realized when R₃ is C₁₋₆ alkyl. A preferred alkyl is isopropyl.

Another embodiment of this invention is realized when R₂ is COOR₃ and all other variables are as originally defined. A sub-embodiment of this invention is realized when R₃ is hydrogen. Another sub-embodiment of this invention is realized when R₃ is C₁₋₆ alkyl, preferably isopropyl.

Still another embodiment of this invention is realized when R₂ is a carboxylic acid esostere and all other variables are as originally defined. A sub-embodiment of this invention is realized when the carboxylic esostere is tetrazole.

Another embodiment of this invention is realized when the (CH₂)ₙC₃₋₁₀ cycloalkyl; —(CH₂)ₙC₃₋₁₀ heterocyclyl, (CH₂)ₙC₆₋₁₀ aryl groups of R is selected from the group consisting of

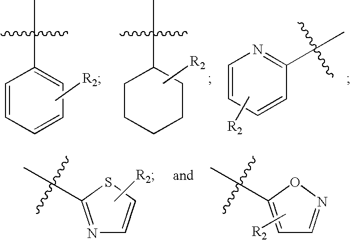

and all other variables are as originally described.

Another embodiment of this invention is realized when R is (CH₂)ₙC₆₋₁₀ aryl which is defined as

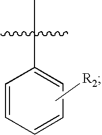

and all other variables are as originally defined. A sub-embodiment of this invention is realized when R₂ is COOH, or COOC₁₋₆ alkyl, preferably the alkyl is isopropyl. Another sub-embodiment is realized when R₂ is a carboxylic acid esostere, preferably the esostere is tetrazole.

Another embodiment of this invention is realized when R is (CH₂)ₙC₃₋₁₀ cycloalkyl which is defined as

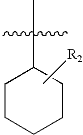

and all other variables are as originally defined. A sub-embodiment of this invention is realized when R₂ is COOH, or COOC₁₋₆ alkyl, preferably the alkyl is isopropyl. Another sub-embodiment is realized when R₂ is a carboxylic acid esostere, preferably the esostere is tetrazole.

Another embodiment of this invention is realized when R is (CH₂)ₙC₃₋₁₀ heterocyclyl, which is defined as

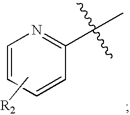

and all other variables are as originally defined. A sub-embodiment of this invention is realized when R₂ is COOH, or COOC₁₋₆ alkyl, preferably the alkyl is isopropyl. Another sub-embodiment is realized when R₂ is a carboxylic acid esostere, preferably the esostere is tetrazole.

Another embodiment of this invention is realized when R is (CH₂)ₙC₃₋₁₀ heterocyclyl which is defined as

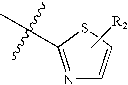

and all other variables are as originally defined. A sub-embodiment of this invention is realized when R₂ is COOH, or COOC₁₋₆ alkyl, preferably the alkyl is isopropyl. Another sub-embodiment is realized when R₂ is a carboxylic acid esostere, preferably the esostere is tetrazole.

Another embodiment of this invention is realized when R is (CH₂)ₙC₃₋₁₀ heterocyclyl which is defined as

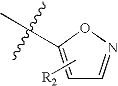

and all other variables are as originally defined. A sub-embodiment of this invention is realized when R₂ is COOH, or COOC₁₋₆ alkyl, preferably the alkyl is isopropyl. Another sub-embodiment is realized when R₂ is a carboxylic acid esostere, preferably the esostere is tetrazole.

Still another embodiment of this invention is realized when R₁ is halogen and all other variables are as originally defined.

Still another embodiment of this invention is realized when R₁ is C₁₋₆ alkyl and all other variables are as originally defined.

Still another embodiment of this invention is realized when R₁ is CF₃ and all other variables are as originally defined.

Another embodiment of this invention is realized when R₁ is bromine or chlorine, preferably bromine and all other variables are as originally defined.

Another embodiment of this invention is realized when n is 0, 1 or 2 and all other variables are as originally defined. A sub-embodiment of this invention is realized when n is 0. Another sub-embodiment is realized when n is 1. Still another sub-embodiment is realized when n is 2.

Another embodiment of this invention is realized when --- represents a double bond.

Another embodiment of this invention is realized when R is (CH₂)ₙC₆₋₁₀ aryl which is

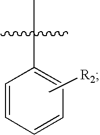

R₂ is COOH, COOCH(CH₃)₂, or tetrazolyl, and R₁ is halogen.

Another embodiment of this invention is realized when R is (CH₂)ₓCOOR₃, x is 3-4, R₁ is halogen and R₃ is COOH. Another embodiment of this invention is realized when R₃ is COOCH(CH₃)₂.

Compounds of this invention are:
Isopropyl 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoic acid;
Isopropyl 4-(2-{(4S)-4-[(3R)-4-(3-bromophenyl)-4,4-difluoro-3-hydroxybutyl]-2-oxo-1,3-oxazinan-3-yl}ethyl)benzoate;
4-(2-{(4S)-4-[(3R)-4-(3-bromophenyl)-4,4-difluoro-3-hydroxybutyl]-2-oxo-1,3-oxazinan-3-yl}ethyl)benzoic acid;
Isopropyl 4-[2-((4S)-4-{(3R)-4,4-difluoro-3-hydroxy-4-[3-(trifluoromethyl)phenyl]butyl}-2-oxo-1,3-oxazinan-3-yl)ethyl]benzoate;
4-[2-((4S)-4-{(3R)-4,4-difluoro-3-hydroxy-4-[3-(trifluoromethyl)phenyl]butyl}-2-oxo-1,3-oxazinan-3-yl)ethyl]benzoic acid;
Isopropyl 4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoate;
4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoic acid;
Isopropyl 4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoic acid;
4-(2-{(4S)-4-[(3R)-4-(3,5-dimethylphenyl)-4,4-difluoro-3-hydroxybutyl]-2-oxo-1,3-oxazinan-3-yl}ethyl)benzoic acid;
4-(2-{(4S)-4-[(3R)-4,4-difluoro-3-hydroxy-4-phenylbutyl]-2-oxo-1,3-oxazinan-3-yl}ethyl)benzoic acid;
Isopropyl 4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoic acid;
4-(2-{(4R)-4-[(1E,3R)-4-(3-bromophenyl)-4,4-difluoro-3-hydroxybut-1-en-1-yl]-2-oxo-1,3-oxazinan-3-yl}ethyl)cyclohexanecarboxylic acid;
4-(2-{(4S)-4-[(3R)-4-(3-bromophenyl)-4,4-difluoro-3-hydroxybutyl]-2-oxo-1,3-oxazinan-3-yl}ethyl)cyclohexanecarboxylic acid;
7-{(4R)-4-[(1E,3R)-4-(3-bromophenyl)-4,4-difluoro-3-hydroxybut-1-en-1-yl]-2-oxo-1,3-oxazinan-3-yl}heptanoic acid;
7-{(4S)-4-[4-(3-bromophenyl)-4,4-difluoro-3-hydroxybutyl]-2-oxo-1,3-oxazinan-3-yl}heptanoic acid;
7-{(4R)-4-[(1E,3R)-4-biphenyl-3-yl-4,4-difluoro-3-hydroxybut-1-en-1-yl]-2-oxo-1,3-oxazinan-3-yl}heptanoic acid;
7-{(4R)-4-[(1E,3R)-4,4-difluoro-3-hydroxy-4-(2′-methylbiphenyl-3-yl)but-1-en-1-yl]-2-oxo-1,3-oxazinan-3-yl}heptanoic acid;
Methyl 4-(3-{(4R)-4-[(1E,3R)-4-(3-bromophenyl)-4,4-difluoro-3-hydroxybut-1-en-1-yl]-2-oxo-1,3-oxazinan-3-yl}propyl)benzoate;
4-(3-{(4R)-4-[(1E,3R)-4-(3-bromophenyl)-4,4-difluoro-3-hydroxybut-1-en-1-yl]-2-oxo-1,3-oxazinan-3-yl}propyl)benzoic acid;
Methyl 6-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)pyridine-2-carboxylate;
6-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)pyridine-2-carboxylic acid;
Methyl 2-(3-{(4R)-4-[(1E,3R)-4-(3-bromophenyl)-4,4-difluoro-3-hydroxybut-1-en-1-yl]-2-oxo-1,3-oxazinan-3-yl}propyl)-1,3-thiazole-5-carboxylate;
2-(3-{(4R)-4-[(1E,3R)-4-(3-bromophenyl)-4,4-difluoro-3-hydroxybut-1-en-1-yl]-2-oxo-1,3-oxazinan-3-yl}propyl)-1,3-thiazole-5-carboxylic acid;
3-(3-{(4R)-4-[(1E,3R)-4-(3-bromophenyl)-4,4-difluoro-3-hydroxybut-1-en-1-yl]-2-oxo-1,3-oxazinan-3-yl}propyl)benzoic acid;
3-(3-{(4S)-4-[(3R)-4-(3-bromophenyl)-4,4-difluoro-3-hydroxybutyl]-2-oxo-1,3-oxazinan-3-yl}propyl)benzoic acid; and
5-(3-{(4R)-4-[(1E,3R)-4-(3-bromophenyl)-4,4-difluoro-3-hydroxybut-1-en-1-yl]-2-oxo-1,3-oxazinan-3-yl}propyl)isoxazole-3-carboxylic acid;
   or a pharmaceutically acceptable salt, enantiomer, diastereomer, prodrug or mixture thereof.

Preferred compounds are
Isopropyl 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoic acid;
Isopropyl 4-(2-{(4S)-4-[(3R)-4-(3-bromophenyl)-4,4-difluoro-3-hydroxybutyl]-2-oxo-1,3-oxazinan-3-yl}ethyl)benzoate;
4-(2-{(4S)-4-[(3R)-4-(3-bromophenyl)-4,4-difluoro-3-hydroxybutyl]-2-oxo-1,3-oxazinan-3-yl}ethyl)benzoic acid;
Isopropyl 4-[2-((4S)-4-{(3R)-4,4-difluoro-3-hydroxy-4-[3-(trifluoromethyl)phenyl]butyl}-2-oxo-1,3-oxazinan-3-yl)ethyl]benzoate;
4-[2-((4S)-4-{(3R)-4,4-difluoro-3-hydroxy-4-[3-(trifluoromethyl)phenyl]butyl}-2-oxo-1,3-oxazinan-3-yl)ethyl]benzoic acid;
Isopropyl 4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoate;
4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoic acid;
Isopropyl 4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoic acid;
4-(2-{(4S)-4-[(3R)-4-(3,5-dimethylphenyl)-4,4-difluoro-3-hydroxybutyl]-2-oxo-1,3-oxazinan-3-yl}ethyl)benzoic acid;
4-(2-{(4S)-4-[(3R)-4,4-difluoro-3-hydroxy-4-phenylbutyl]-2-oxo-1,3-oxazinan-3-yl}ethyl)benzoic acid;
Isopropyl 4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoic acid;
   or a pharmaceutically acceptable salt, enantiomer, diastereomer, prodrug or mixture thereof.

Another embodiment of this invention is directed to a composition containing an EP₄ agonist of Formula I and optionally a pharmaceutically acceptable carrier.

Yet another embodiment of this invention is directed to a method for decreasing elevated intraocular pressure or treating glaucoma by administration, preferably topical or intra-camaral administration, of a composition containing an EP₄ agonist of Formula I and optionally a pharmaceutically acceptable carrier. Use of the compounds of formula I for the manufacture of a medicament for treating elevated intraocular pressure or glaucoma or a combination thereof is also included in this invention

This invention is further concerned with a process for making a pharmaceutical composition comprising a compound of formula I.

This invention is further concerned with a process for making a pharmaceutical composition comprising a compound of formula I, and a pharmaceutically acceptable carrier.

The claimed compounds bind strongly and act on PGE₂ receptor, particularly on the EP₄ subtype receptor and therefore are useful for preventing and/or treating glaucoma and ocular hypertension.

Dry eye is a common ocular surface disease afflicting millions of people. Although it appears that dry eye may result from a number of unrelated pathogenic causes, the common end result is the breakdown of the tear film, which results in dehydration of the exposed outer surface of the eye. (Lemp, Report of the Nation Eye Institute/Industry Workshop on Clinical Trials in Dry Eyes, The CLAO Journal, 21(4):221-231 (1995)). Functional EP4 receptors have been found in human conjunctival epithelial cells (see U.S. Pat. No. 6,344,477, incorporated by reference in its entirety) and it is appreciated that both human corneal epithelial cells (Progress in Retinal and Eye Research, 16:81-98 (1997)) and conjunctival cells (Dartt et al. Localization of nerves adjacent to goblet cells in rat conjunctiva. Current Eye Research, 14:993-1000 (1995)) are capable of secreting mucins. Thus, the compounds of formula I are useful for treating dry eye.

Macular edema is swelling within the retina within the critically important central visual zone at the posterior pole of the eye. It is believed that EP₄ agonist which lower IOP are useful for treating diseases of the macular such as macular edema or macular degeneration. Thus, another aspect of this invention is a method for treating macular edema or macular degeneration.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, January 1994, pp. 37-44; Investigative Ophthamol. & Visual Science, 32, 5, April 1991, pp. 1593-99. It is believed that EP₄ agonist which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, or increasing retinal and optic nerve oxygen tension or providing a neuroprotective effect or a combination thereof by using an EP₄ agonist of formula I.

The compounds produced in the present invention are readily combined with suitable and known pharmaceutically acceptable excipients to produce compositions which may be administered to mammals, including humans, to achieve effective IOP lowering. Thus, this invention is also concerned with compositions and methods of treating ocular hypertension, glaucoma, macular edema, macular degeneration, for increasing retinal and optic nerve head blood velocity, for increasing retinal and optic nerve oxygen tension, for providing a neuroprotective effect or for a combination thereof by administering to a patient in need thereof one of the compounds of formula I alone or in combination with one or more of the following active ingredients, a β-adrenergic blocking agent such as timolol, betaxolol, levobetaxolol, carteolol, levobunolol, a parasympathomimetic agent such as pilocarpine, a sympathomimetic agents such as epinephrine, iopidine, brimonidine, clonidine, para-aminoclonidine, a carbonic anhydrase inhibitor such as dorzolamide, acetazolamide, metazolamide or brinzolamide; COSOPT®, a Maxi-K channel blocker such as Penitrem A, paspalicine, charybdotoxin, iberiotoxin, Paxicillan, Aflitram, Verroculogen, and as disclosed in WO 03/105868 (U.S. Ser. No. 60/389,205), WO 03/105724 (60/389,222), WO 03/105847 (60/458,981), 60/424,790, filed Nov. 8, 2002 (Attorney docket 21260PV), 60/424,808, filed Nov. 8, 2002 (Attorney docket 21281PV), 09/765,716, filed Jan. 17, 2001, 09/764,738, filed Jan. 17, 2001 and PCT publications WO 02/077168 and WO 02/02060863, all incorporated by reference in their entirety herein, and in particular Maxi-K channel blockers such as 1-(1-isobutyl-6-methoxy-1H-indazol-3-yl)-2-methylpropan-1-one; 1-[1-(2,2-dimethylpropyl)-6-methoxy-1H-indazol-3-yl]-2-methylpropan-1-one; 1-[1-(cyclohexylmethyl)-6-methoxy-1H-indazol-3-yl]-2-methylpropan-1-one; 1-(1-hexyl-6-methoxy-1H-indazol-3-yl)-2-methylpropan-1-one; 1-[1-(2-ethylhexyl)-6-methoxy-1H-indazol-3-yl]-2-methylpropan-1-one; 1-(3-isobutyryl-6-methoxy-1H-indazol-1-yl)buan-2-one; 1-(3-isobutyryl-6-methoxy-1H-indazol-1-yl)-3,3-dimethylbutan-2-one; 1-(3-cyclopentylcarbonyl)-6-methoxy-1H-indazol-1-yl)-3,3-dimethylbutan-2-one; 1-(3,3-dimethyl-2-oxobutyl)-6-methoxy-1H-indazole-3-carboxylic acid; and 1-[3-(3-hydroxypropanoyl)-6-methoxy-1H-indazol-1-yl]-3,3-dimethylbutan-2-one, a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; and/or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine.

Use of the compounds of formula I for the manufacture of a medicament for treating ocular hypertension, glaucoma, macular edema, macular degeneration, for increasing retinal and optic nerve head blood velocity, for increasing retinal and optic nerve oxygen tension, for providing a neuroprotective effect or for a combination thereof is also included in this invention.

The EP₄ agonist used in the instant invention can be administered in a therapeutically effective amount intravenously, subcutaneously, topically, transdermally, parenterally or any other method known to those skilled in the art. Ophthalmic pharmaceutical compositions are preferably adapted for topical administration to the eye in the form of solutions, suspensions, ointments, creams or as a solid insert. Ophthalmic formulations of this compound may contain from 0.00001 to 0.5% and especially 0.00005 to 0.1% of medicament. Higher dosages as, for example, up to about 10% or lower dosages can be employed provided the dose is effective in reducing intraocular pressure, treating glaucoma, increasing blood flow velocity or oxygen tension. For a single dose, from between 0.000001 to 0.05 mg, preferably 0.000005 to 0.01 mg, and especially 0.00005 to 0.005 mg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, polysorbate-80, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats, rabbits and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mammalian eye will be from once up to three times daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The compounds of the instant invention are also useful for mediating the bone modeling and remodeling processes of the osteoblasts and osteoclasts. See PCT US99/23757 filed Oct. 12, 1999 and incorporated herein by reference in its entirety. The major prostaglandin receptor in bone is EP₄, which is believed to provide its effect by signaling via cyclic AMP. See Ikeda T, Miyaura C, Ichikawa A, Narumiya S, Yoshiki S and Suda T 1995, In situ localization of three subtypes (EP₁, EP₃ and EP₄) of prostaglandin E receptors in embryonic and newborn mice, J Bone Miner Res 10 (sup 1): S172, which is incorporated by reference herein in its entirety. Use of the compounds of formula I for the manufacture of a medicament for mediating the bone modeling and remodeling processes are also included in this invention.

Thus, another object of the present invention is to provide methods for stimulating bone formation, i.e. osteogenesis, in a mammal comprising administering to a mammal in need thereof a therapeutically effective amount of an EP₄ receptor subtype agonist of formula I.

Still another object of the present invention to provide methods for stimulating bone formation in a mammal in need thereof comprising administering to said mammal a therapeutically effective amount of an EP₄ receptor subtype agonist of formula I and a bisphosphonate active. Use of the compounds of formula I for the manufacture of a medicament for stimulating bone formation is also included in this invention.

Yet another object of the present invention to provide pharmaceutical compositions comprising a therapeutically effective amount of an EP₄ receptor subtype agonist of formula I and a bisphosphonate active.

It is another object of the present invention to provide methods for treating or reducing the risk of contracting a disease state or condition related to abnormal bone resorption in a mammal in need of such treatment or prevention, comprising administering to said mammal a therapeutically effective amount of an EP₄ receptor subtype agonist of formula I. Use of the compounds of formula I for the manufacture of a medicament for treating or reducing the risk of contracting a disease state or condition related to abnormal bone resorption is also included in this invention.

The disease states or conditions related to abnormal bone resorption include, but are not limited to, osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy, and multiple myeloma.

Within the method comprising administering a therapeutically effective amount of an EP₄ receptor subtype agonist of formula I and a bisphosphonate active, both concurrent and sequential administration of the EP₄ receptor subtype agonist of formula I and the bisphosphonate active are deemed within the scope of the present invention. Generally, the formulations are prepared containing 5 or 10 mg of a bisphosphonate active, on a bisphosphonic acid active basis. With sequential administration, the agonist and the bisphosphonate can be administered in either order. In a subclass of sequential administration the agonist and bisphosphonate are typically administered within the same 24 hour period. In yet a further subclass, the agonist and bisphosphonate are typically administered within about 4 hours of each other.

A non-limiting class of bisphosphonate actives useful in the instant invention are selected from the group consisting of alendronate, cimadronate, clodronate, tiludronate, etidronate, ibandronate, neridronate, olpandronate, risedronate, piridronate, pamidronate, zolendronate, pharmaceutically acceptable salts thereof, and mixtures thereof.

A non-limiting subclass of the above-mentioned class in the instant case is selected from the group consisting of alendronate, pharmaceutically acceptable salts thereof, and mixtures thereof.

A non-limiting example of the subclass is alendronate monosodium trihydrate.

In the present invention, as it relates to bone stimulation, the agonist is typically administered for a sufficient period of time until the desired therapeutic effect is achieved. The term “until the desired therapeutic effect is achieved”, as used herein, means that the therapeutic agent or agents are continuously administered, according to the dosing schedule chosen, up to the time that the clinical or medical effect sought for the disease or condition being mediated is observed by the clinician or researcher. For methods of treatment of the present invention, the compounds are continuously administered until the desired change in bone mass or structure is observed. In such instances, achieving an increase in bone mass or a replacement of abnormal bone structure with normal bone structure are the desired objectives. For methods of reducing the risk of a disease state or condition, the compounds are continuously administered for as long as necessary to prevent the undesired condition. In such instances, maintenance of bone mass density is often the objective.

Nonlimiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

The instant compounds are also useful in combination with known agents useful for treating or preventing bone loss, bone fractures, osteoporosis, glucocorticoid induced osteoporosis, Paget's disease, abnormally increased bone turnover, periodontal disease, tooth loss, osteoarthritis, rheumatoid arthritis, periprosthetic osteolysis, osteogenesis imperfecta, metastatic bone disease, hypercalcemia of malignancy, and multiple myeloma. Combinations of the presently disclosed compounds with other agents useful in treating or preventing osteoporosis or other bone disorders are within the scope of the invention. A person of ordinary skill in the art would be able to discern which combinations of agents would be useful based on the particular characteristics of the drugs and the disease involved. Such agents include the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; and the pharmaceutically acceptable salts and mixtures thereof. A preferred combination is a compound of the present invention and an organic bisphosphonate. Another preferred combination is a compound of the present invention and an estrogen receptor modulator. Another preferred combination is a compound of the present invention and an estrogen. Another preferred combination is a compound of the present invention and an androgen receptor modulator. Another preferred combination is a compound of the present invention and an osteoblast anabolic agent.

Regarding treatment of abnormal bone resorption and ocular disorders, the formula I agonists generally have an EC₅₀ value from about 0.001 nM to about 100 microM, although agonists with activities outside this range can be useful depending upon the dosage and route of administration. In a subclass of the present invention, the agonists have an EC₅₀ value of from about 0.0001 microM to about 10 microM. In a further subclass of the present invention, the agonists have an EC₅₀ value of from about 0.001 microM to about 0.1 microM. EC₅₀ is a common measure of agonist activity well known to those of ordinary skill in the art and is defined as the concentration or dose of an agonist that is needed to produce half, i.e. 50%, of the maximal effect. See also, Goodman and Gilman's, The Pharmacologic Basis of Therapeutics, 9th edition, 1996, chapter 2, E. M. Ross, Pharmacodynamics, Mechanisms of Drug Action and the Relationship Between Drug Concentration and Effect, and PCT US99/23757, filed Oct. 12, 1999, which are incorporated by reference herein in their entirety.

The herein examples illustrate but do not limit the claimed invention. Each of the claimed compounds are EP₄ agonists and are useful for a number of physiological ocular and bone disorders.

The compounds of this invention can be made, with some modification, in accordance with U.S. Pat. No. 6,043,275, EP0855389, WO 03/047417 (U.S. Ser. No. 60/337,228), WO 03/047513 (U.S. Ser. No. 60/338,117), U.S. Ser. No. 60/406,530 (Merck Docket No. MC060), WO 2004/085430 and WO 01/46140, all of which are incorporated herein by reference in their entirety. The following non-limiting schemes and examples given by way of illustration is demonstrative of the present invention.

The preparation of compounds from the current invention can be accomplished according to general schemes 1 through 4, and is further illustrated in the experimental section.

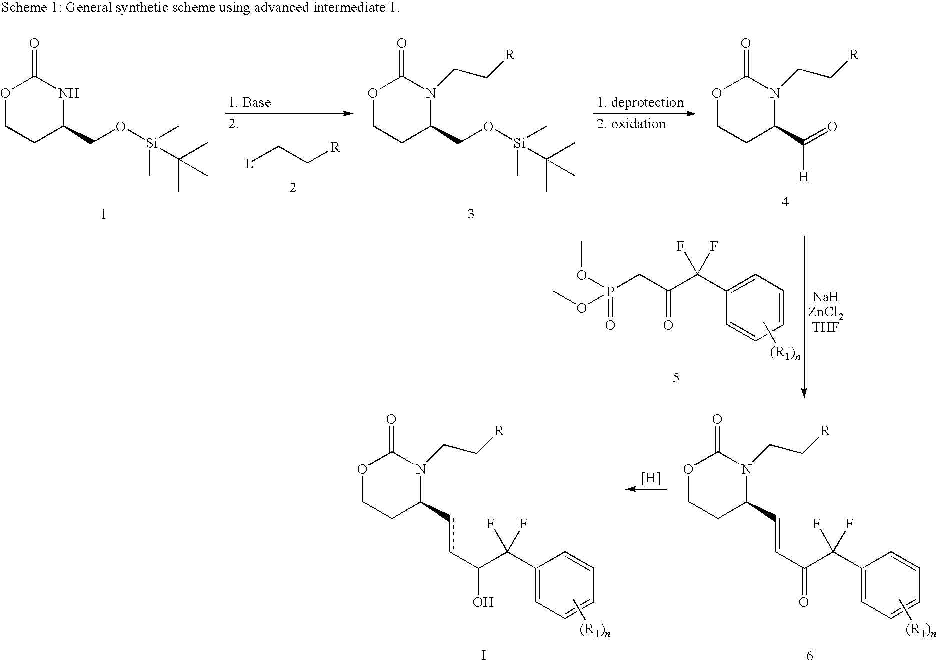

(4R)-4-({[tert-butyl(dimethyl)silyl]oxy}methyl)-1,3-oxazinan-2-one (1) (WO 2004/085430) was first treated with a strong base such as potassium hexamethyldisilazide (KHMDS) or NaH followed by treating with reagent 2 (L=I, MeSO₂O, Br, etc.) in either THY (tetrahydrofuran) or DMF (dimethylformamide) to give the alkylation product 3. Deprotection of the t-butyldimethylsilyl (TBS) group with 1N aqueous HCl or with TBAF followed by oxidation of the resultant alcohol with a suitable oxidant gave aldehyde 4.4 was then reacted with reagent 5 using NaH as the base and ZnCl₂ as the Lewis acid to give ketone 6. Reduction of 6 with suitable reducing reagents gave I.

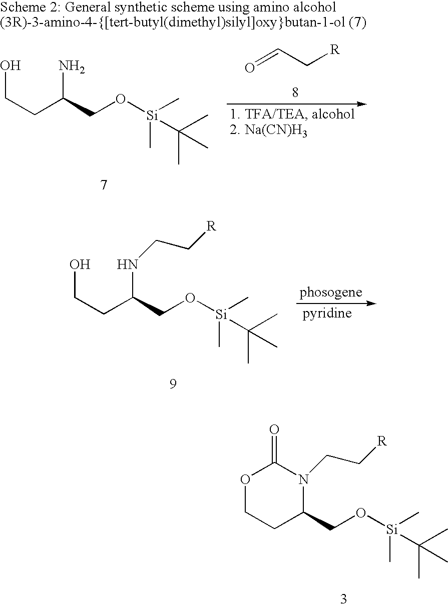

Reductive amination of amino alcohol 7 (WO 2004/085430) with a suitable aldehyde 8 followed by cyclization with phosgene using pyridine as the base gave 1,3-oxazinane intermediate 3. The intermediate can be processed to the desired product according to Scheme 1.

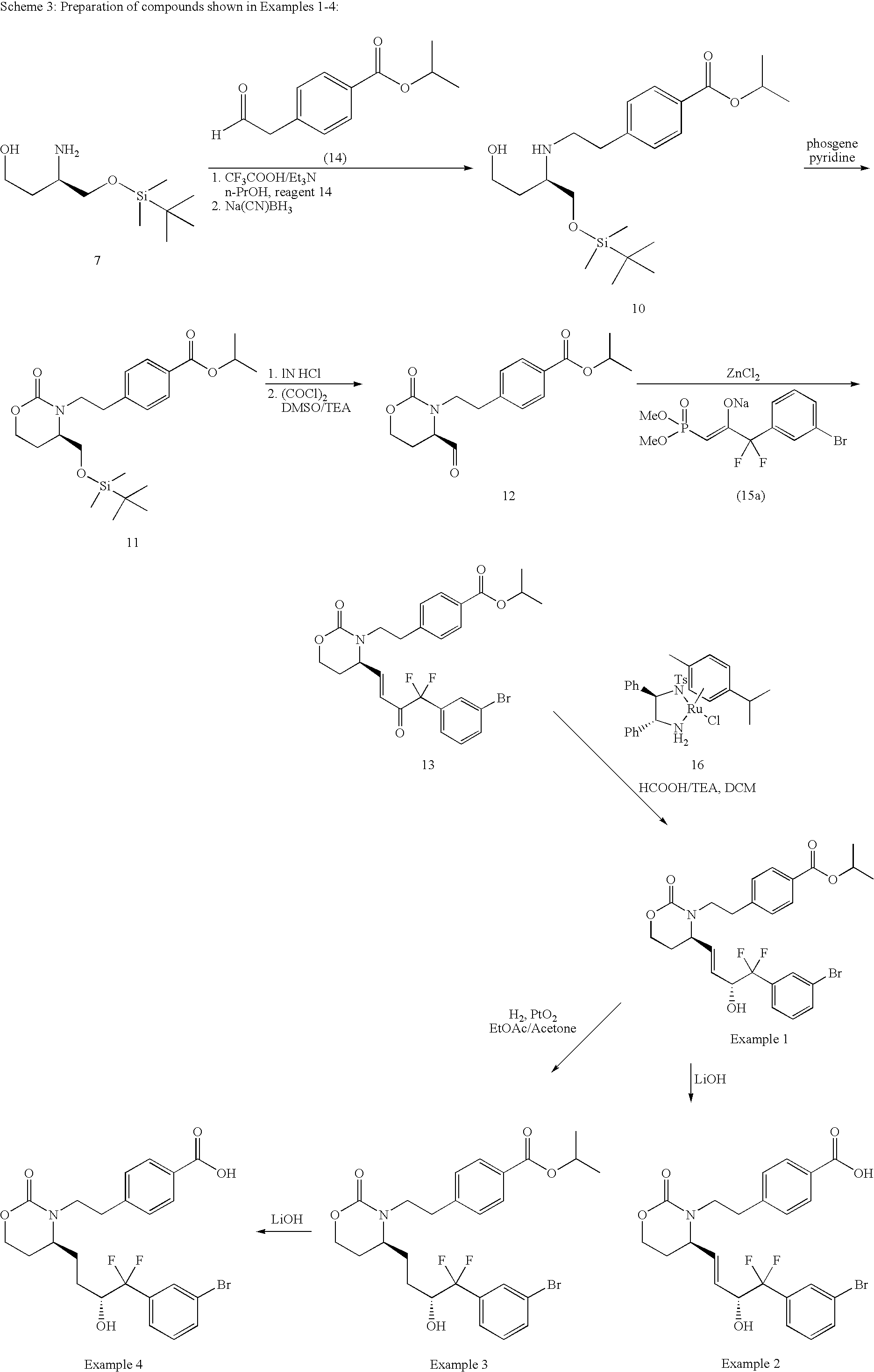

1. Preparation of isopropyl 4-(2-oxoethyl)benzoate (14)—The synthesis of aldehyde 14 is illustrated in Scheme 4.

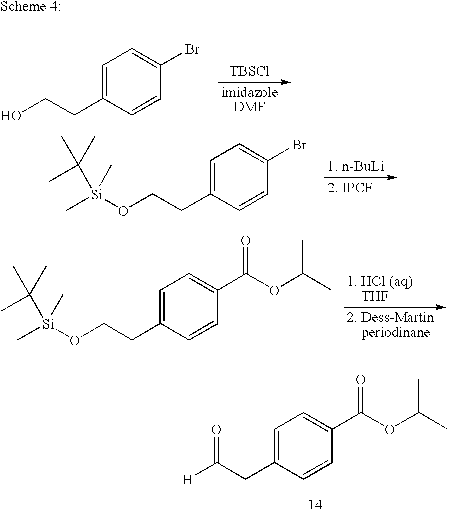

### Step 1: [2-(4-bromophenyl)ethoxy](tert-butyl)dimethylsilane

To a solution of 2-(4-bromophenyl)ethanol (5.3 g, 26.4 mmol) in DMF (50 mL) at 0° C. was added imidazole (3.59 g, 52.8 mmol, 2 eq) and tert-Butyldimethylsilyl chloride (TBSCl) (4.18 g, 27.7 mmol, 1.05 eq) and the mixture was stirred at 0° C. until all starting material was consumed. The mixture was then diluted with water and extracted with ether (3×). The extracts were washed with water (3×) and brine and dried over MgSO₄ to give the desired product. ¹H NMR (400 MHz, acetone-d₆) δ 7.46 (2H, d, J=8 Hz), 7.22 (2h, D, j=8 Hz), 3.85 (t, 2H, J=7 Hz), 2.79 (t, 2H, J=7 Hz), 0.87 (s, 9H) and -0.01 (s, 6H).

### Step 2: isopropyl 4-(2-{[tert-butyl(dimethyl)silyl]oxy}ethyl)benzoate

To a solution of [2-(4-bromophenyl)ethoxy](tert-butyl)dimethylsilane (7.3 g, 23.15 mmol) in THF at −78° C. was added n-Butyllithium (2.5M in hexanes, 10.2 mL, 25.5 mmol, 1.1 eq) dropwise and the mixture was stirred at the temperature for 10 min. The solution was then added to a THF solution of isopropyl chloroformate (IPCF, 1.0M solution in toluene, 46.3 mL, 46.3 mmol, 2 eq) via a cannula at −78° C. and the mixture was stirred for 30 min at −78° C. and quenched with saturated NaHCO₃. Worked up as usual followed by flash chromatography purification gave the desired product. ¹H NMR δ (ppm)(Acetone-d₆): 7.94 (2H, d, J=8.2 Hz), 7.39 (2H, d, J=8.2 Hz), 5.24-5.16 (1H, m), 3.91-3.85 (2H, m), 2.89 (2H, t, J=6.5 Hz), 1.36 (6H, d, J=6.2 Hz), 0.87 (9H, s), −0.01 (5H, s).

### Step 3: isopropyl 4-(2-hydroxyethyl)benzoate

A mixture isopropyl 4-(2-{[tert-butyl(dimethyl)silyl]oxy}ethyl)benzoate (7.3 g, 22.63 mmol) and 1N HCl (24.89 mL, 24.89 mmol, 1.1 eq) in THF (75 mL) was stirred at rt until all starting material disappeared (approx. 1 h). The mixture was concentrated and the residue extracted with EA. The crude was purified by flash (30-40% EA/hex) to give the desired alcohol. ¹H NMR δ (ppm)(Acetone-d₆): 7.93 (2H, d, J=8.2 Hz), 7.39 (2H, d, J=8.2 Hz), 5.23-5.15 (1H, m), 3.83-3.75 (3H, m), 2.90 (3H, d, J=6.4 Hz), 1.35 (6H, d, J=6.2 Hz).

### Step 4: isopropyl 4-(2-oxoethyl)benzoate (14)

To a solution of isopropyl 4-(2-hydroxyethyl)benzoate (2 g, 9.6 mmol) in DCM was added Dess-Martin periodinane (4.28 g, 10.08 mmol, 1.05 eq) and the mixture was stirred at rt for 30 min (slight exotherm) and then concentrated in vacuo. The residue was resuspended in ether and filtered. The filtrate was concentrated to give the crude product (14) which was co-evaporated with toluene (2×) and pumped under high vacuum to remove AcOH. The crude product was used directly without further purification. ¹H NMR δ (ppm)(CDCl₃): 9.78 (1H, s), 8.06 (2H, d, J=8.2 Hz), 7.31 (2H, d, J=8.1 Hz), 5.31-5.23 (1H, m), 3.79 (2H, d, J=1.7 Hz), 1.39 (6H, d, J=6.2 Hz).

### 2: Preparation of Horner-Wordsworth-Emmons Reagents 15 is Outlined in Scheme 5.

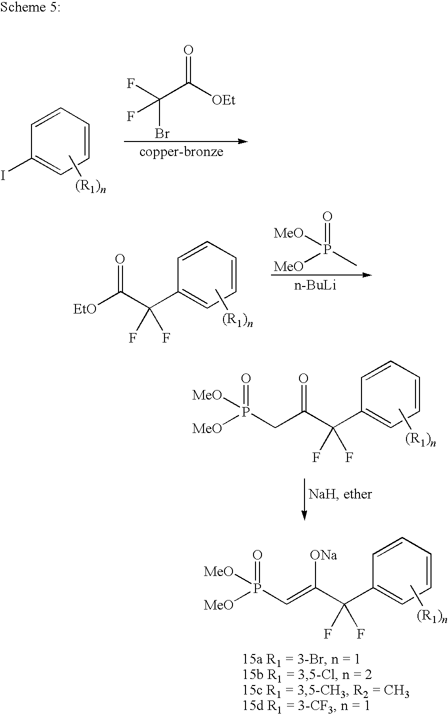

### Preparation of Reagent 15a

Step 1: To a solution of 3-bromo-iodobenzene (14.1 g, 50 mmol) and ethyl bromo-α,α-difluoroacetate (10.1 g, 50 mmol) in DMSO (40 mL) was added copper bronze (7 g, 110 mmol) and the suspension was heated to 55° C. for 2.5 d and cooled to rt. The mixture was quenched with KH₂PO₄ and filtered. The solid was washed with EA/water and the filtrated was separated. The aqueous layer was extracted with ether (2×) and the organic phases were combined, washed with water and brine. The crude was purified by flash chromatography (5-10% EA/hex) to give 10.7 g desired product as a colorless oil.

To a solution of dimethyl methylphosphonate (4.1 g, 33 mmol) in THF (100 mL) at −78° C. was added n-BuLi (12.6 mL, 2.5M in hexanes) dropwise and the mixture was stirred at the temperature for 1 h. To this solution was then added ethyl α,α-difluoro-3-bromophenylacetate (8.37 g, 30 mmol) in THF via a cannula and the mixture was stirred at −78° C. for 2 h and quenched with 2.2 mL AcOH and water. After warming to rt, the mixture was extracted with EA (3×). The organic layers were washed with brine, dried over sodium sulfate, filtered and concentrated in vacuo to give the desired product dimethyl [3-(3-bromophenyl)-3,3-difluoro-2-oxopropyl]phosphonate as a colorless oil. To a solution of this oil (8.28 g, 23.19 mmol) in ether at rt was added sodium hydride 60% (974 mg, 24.35 mmol, 1.05 eq) portionwise and the white suspension stirred at rt for 1 h. The mixture was filtered and the white solid washed with ether/hexane. The solid thus obtained was dried under high vacuum to give 15a (white powder). Reagents 15b-15d were prepared in a similar manner.

### 3: Preparation of Catalyst 16

The catalyst was prepared by mixing 1 mol equiv of [RuCl₂(p-cymene)₂], 2 mol equiv (R,R)—N-Tosyl-1,2-diphenylethylene-1,2-diamine and 4.2 mol equiv of Et₃N in iPrOH at 80° C. for 1 h (hour). After solvent removal, the solid was washed with cold H₂O and the recrystallized from MeOH to give the catalyst as orange solid.

The catalyst could also be generated in situ by mixing 0.02 mol equiv of [RuCl₂(p-cymene)₂] and 0.04 mol equiv of the (R,R)—N-Tosyl-1,2-diphenylethylene-1,2-diamine in DCM (dichloromethane) in the presence of 0.04 mol equiv of 1M solution KOtBu in THF. After aging for 10 min at RT (room temperature), Et₃N was added followed by HCO₂H and a solution of the enone in DCM.

### Example 1

### isopropyl 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoate

### Step 1: isopropyl 4-(2-{[(1R)-1-({[tert-butyl(dimethyl)silyl]oxy}methyl)-3-hydroxypropyl]amino}ethyl)benzoate (10)

To a solution of (3R)-3-amino-4-{[tert-butyl(dimethyl)silyl]oxy}butan-1-ol (7) (see WO 2004/085430) (1.7 g, 7.75 mmol) in n-PrOH at −10° C. was added trifluoroacetic acid (597 uL, 7.75 mmol, 1 eq) followed by triethylamine (981 uL, 6.98 mmol, 0.9 eq). The solution was stirred for 1 min. isopropyl 4-(2-oxoethyl)benzoate (14) (2.24 g, 10.85 mmol, 1.4 eq) was then added and the mixture was stirred at 5° C. overnight (o/n). Sodium cyanoborohydride (730 mg, 11.62 mmol, 1.5 eq) was then added in one portion at 0° C. and the mixture was stirred at 0° C. for 2 h and quenched with saturated NH₄Cl and then treated with NaHCO₃. The mixture was then extracted with DCM (3×). The extracts were dried over Na₂SO₄, filtered and concentrated. The crude was purified by flash chromatography (5-10% MeOH/DCM with 1% TEA) to give the desired product 10. MS (+ESI): m/z 410.5 (M+1)⁺.

### Step 2: isopropyl 4-{2-[(4R)-4-({[tert-butyl(dimethyl)silyl]oxy}methyl)-2-oxo-1,3-oxazinan-3-yl]ethyl}benzoate (11)

To a solution of crude isopropyl 4-(2-{[(1R)-1-({[tert-butyl(dimethyl)silyl]-oxy}methyl)-3-hydroxypropyl]amino}ethyl)benzoate (10) (1.5 g, 3.66 mmol) in DCM was added pyridine (888 uL, 10.98 mmol, 3 eq) followed by phosgene solution (20% in toluene, 3.4 mL, 7 mmol, 1.2 eq) dropwise and the mixture was stirred 0° C. for 1 h and warmed to rt for 30 min. The mixture was washed with 1N HCl and dried over MgSO₄. The crude was purified by combi-flash (20-70% EA/hex in 15 min) to give the desired product 11 as a light yellow viscous oil. MS (+ESI): 436.4 (M+1)⁺. ¹H NMR δ (ppm)(Acetone-d₆): 7.97 (2H, d, J=8.1 Hz), 7.40 (2H, d, J=8.1 Hz), 5.24-5.16 (1H, m), 4.36-4.30 (1H, m), 4.15-4.07 (1H, m), 3.77-3.65 (3H, m), 3.44-3.34 (2H, m), 2.03-1.93 (2H, m), 1.36 (6H, d, J=6.2 Hz), 0.89 (9H, s), 0.08 (6H, s).

### Step 3: isopropyl 4-{2-[(4R)-4-formyl-2-oxo-1,3-oxazinan-3-yl]ethyl}benzoate (12)

The mixture of 11 (1.2 g, 2.75 mmol) and 1N HCl (11 mL, 11 mmol, 4 eq) in THF (40 mL) was stirred at rt o/n and concentrated. The residue was redissolved in EA and washed with brine, dried over Na₂SO₄ and filtered. The filtrate was concentrated to give 0.86 g crude alcohol which was used directly without further purification. ¹H NMR δ (ppm)(Acetone-d₆): 7.96 (2H, d, J=8.1 Hz), 7.41 (2H, d, J=8.1 Hz), 5.24-5.16 (1H, m), 4.37-4.31 (1H, m), 4.17-4.09 (2H, m), 3.79-3.65 (3H, m), 3.42-3.32 (2H, m), 3.12-3.06 (1H, m), 2.99-2.93 (1H, m), 2.04 (1H, m), 1.99-1.92 (1H, m), 1.36 (6H, d, J=6.2 Hz). To a solution of DMSO (Dimethylsulfoxide) (229 uL, 3.22 mmol, 1.2 eq) in CH₂Cl₂ (DCM) (10 mL) at −78° C. was added oxalyl chloride (258 uL, 2.95 mmol, 1.1 eq) dropwise and the mixture was stirred at the temperature for 15 min. To this mixture was added a solution of the alcohol from above (0.86 g, 2.68 mmol) in DCM (5 mL) via a cannula and the resultant mixture was stirred at −78° C. for 15 min. Triethylamine (942 uL, 6.7 mmol, 2.5 eq) was then introduced using a syringe and the mixture was stirred at −78° C. for 30 min before warming to 0° C. in air. The volatiles were removed in vacuo and the residue resuspended in ether/ethyl acetate and filtered. The filtrate was concentrated in vacuo to give the desired aldehyde 12. ¹H NMR δ (ppm)(CDCl₃): 9.52 (1H, s), 7.99 (2H, d, J=8.2 Hz), 7.30 (2H, d, J=8.2 Hz), 5.31-5.21 (1H, m), 4.22-4.18 (1H, m), 4.10-3.98 (2H, m), 3.59 (1H, dd, J=3.3, 6.5 Hz), 3.17-3.07 (2H, m), 3.01-2.93 (1H, m), 2.18-2.08 (2H, m), 1.38 (6H, d, J=6.2 Hz).

### Step 4: isopropyl 4-(2-{(4R)-4-[(1E)-4-(3-bromophenyl)-4,4-difluoro-3-oxobut-1-en-1-yl]-2-oxo-1,3-oxazinan-3-yl}ethyl)benzoate (13)

A mixture of aldehyde 12 (0.4 g, 1.25 mmol), reagent 15a (618 mg, 1.63 mmol, 1.3 eq) and zinc chloride (0.5M in THF, 2.76 mL, 1.38 mmol, 1.1 eq) was heated to 60-70° C. overnight (o/n) under N₂ and cooled to rt. The mixture was quenched with NH₄Cl/water (1:1) and extracted with EA (3×). The organic layers were combined, washed with water and brine, dried over MgSO₄ and filtered. The filtrate was concentrated in vacuo to give the crude product which was purified by flash chromatography (40-90% EA/hex) to give product 13. ¹H NMR δ (ppm)(Acetone-d₆): 7.94 (2H, d, J=8.0 Hz), 7.82-7.74 (2 H, m), 7.71-7.51 (2H, m), 7.33 (2H, d, J=8.0 Hz), 7.14-7.06 (1H, m), 6.84 (1H, d, J=15.6 Hz), 5.24-5.16 (1H, m), 4.26 (1H, m), 4.17-4.09 (2H, m), 3.86-3.78 (1H, m), 3.09-3.01 (2H, m), 2.95-2.89 (1H, m), 2.24-2.16 (1H, m), 2.00 (1H, m), 1.36 (6H, d, J=6.2 Hz).

### Example 1

### isopropyl 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoate

To a solution of ketone 13 (0.6 g, 1.844 mmol) in DCM (5 mL) was added formic acid (109 uL, 2.73 mmol, 2.5 eq) and triethylamine (306 uL, 2.18 mmol, 2 eq) followed by Ru catalyst 16 (41 mg). The mixture was stirred at rt for 0.5 h and washed with water. The crude was purified by flash chromatography (50-90% EA/hex) to give 0.38 g product which was repurified by flash chromatography (20-40% acetone/toluene) to give the title compound as a white foamy solid after pumping under high vacuum for 2 days. ¹H NMR δ (ppm)(Acetone-d₆): 7.96 (2H, d, J=8.1 Hz), 7.68 (2H, m), 7.54 (1H, d, J=7.8 Hz), 7.44-7.36 (3H, m), 5.82 (1H, dd, J=6.4, 15.5 Hz), 5.72 (1H, dd, J=5.5, 15.5 Hz), 5.23-5.17 (2H, m), 4.77-4.69 (1H, m), 4.11 (2H, dd, J=2.8, 8.2 Hz), 3.93-3.89 (1H, m), 3.83-3.75 (1H, m), 3.06-2.91 (3H, m), 2.10-2.04 (1H, m), 1.78-1.70 (1H, m), 1.36 (6H, d, J=6.3 Hz).

### Example 2

### 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoic acid

The isopropyl ester from above was treated with LiOH in Methanol/water to give the corresponding acid. ¹H NMR δ (ppm)(Acetone-d₆): 8.00 (2H, d, J=8.2 Hz), 7.68 (2H, m), 7.53 (1H, t, J=9.1 Hz), 7.44-7.38 (3H, m), 5.82 (1H, dd, J=6.1, 15.5 Hz), 5.72 (1H, dd, J=5.4, 15.5 Hz), 5.23 (1H, s), 4.72 (1H, s), 4.10 (2H, m), 3.92-3.88 (1H, m), 3.84-3.74 (1H, m), 3.07-2.92 (4H, m), 2.10-2.02 (1H, m), 1.78-1.70 (1H, m).

### Example 3

### isopropyl 4-(2-{(4S)-4-[(3R)-4-(3-bromophenyl)-4,4-difluoro-3-hydroxybutyl]-2-oxo-1,3-oxazinan-3-yl}ethyl)benzoate

A mixture of isopropyl 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoate (104 mg, 0.188 mmol), platinum(IV) oxide hydrate (9.31 mg, 0.038 mmol, 0.2 eq) in EtOAc (0.5 mL) and acetone (0.5 mL) was evacuated under vacuum and refilled with H₂ (repeated 3×) and then stirred under 1 atm of H₂ for 3 h. The mixture was filtered through a cotton pad and concentrated to give the desired product. ¹H NMR δ (ppm)(Acetone-d₆): 7.95 (2H, d, J=8.1 Hz), 7.69 (2H, m), 7.56 (1H, d, J=7.6 Hz), 7.45 (1H, t, J=7.7 Hz), 7.39 (2H, d, J=8.1 Hz), 5.23-5.15 (1H, m), 4.93 (1H, d, J=6.6 Hz), 4.28-4.22 (1H, m), 4.12-4.02 (2H, m), 3.80-3.72 (1H, m), 3.38 (1H, d, J=3.9 Hz), 3.33-3.25 (1H, m), 3.11-3.03 (1H, m), 2.96-2.85 (1H, m), 1.96-1.74 (4H, m), 1.68-1.60 (1H, m), 1.47-1.39 (1H, m), 1.36 (6H, d, J=6.2 Hz). MS (+APCI): m/z 554.3, 556.2.

### Example 4

### 4-(2-{(4S)-4-[(3R)-4-(3-bromophenyl)-4,4-difluoro-3-hydroxybutyl]-2-oxo-1,3-oxazinan-3-yl}ethyl)benzoic acid

The ester from above was treated with LiOH in Methanol/water to give the corresponding acid. MS (−ESI): m/z 510.2, 512.2.

The following examples were prepared in a similar manner as depicted in Scheme 3.

### Example 5

### isopropyl 4-[2-((4S)-4-{(3R)-4,4-difluoro-3-hydroxy-4-[3-(trifluoromethyl)phenyl]butyl}-2-oxo-1,3-oxazinan-3-yl)ethyl]benzoate

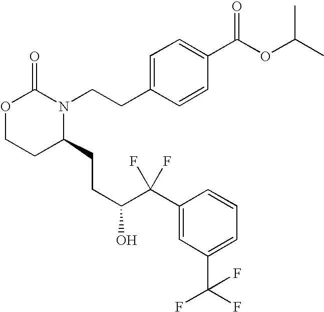

MS (+ESI): m/z 544.2 (M+1)⁺.

### Example 6

### 4-[2-((4S)-4-{(3R)-4,4-difluoro-3-hydroxy-4-[3-(trifluoromethyl)phenyl]butyl}-2-oxo-1,3-oxazinan-3-yl)ethyl]benzoic acid

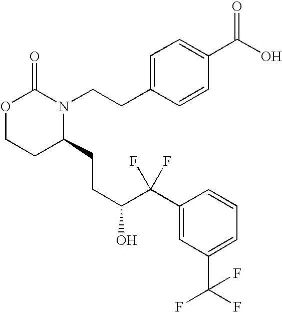

The ester from above was treated with LiOH in Methanol/water to give the corresponding acid. MS (−ESI): m/z 500.1 (M−1)⁻.

### Example 7

### Isopropyl 4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoate

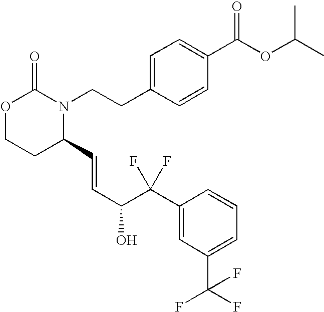

¹H NMR δ (ppm)(Acetone-d₆): 7.98 (2H, d, J=8.2 Hz), 7.86-7.84 (3H, m), 7.73-7.70 (1H, m), 7.36 (2H, d, J=8.2 Hz), 5.88-5.73 (2H, m), 5.31-5.27 (1H, m), 5.23-5.17 (1H, m), 4.81-4.75 (1H, m), 4.14-4.04 (2H, m), 3.96-3.91 (1H, m), 3.83-3.73 (1H, m), 3.07-2.90 (3H, m), 2.12-2.02 (1H, m), 1.77-1.70 (1H, m), 1.36 (6H, d, J=6.3 Hz).

### Example 8

### 4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoic acid

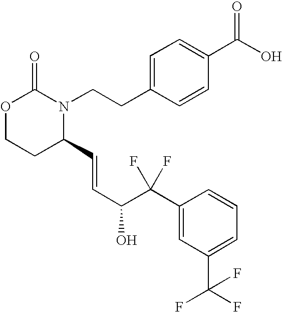

The ester from above was treated with LiOH in Methanol/water to give the corresponding acid. MS (−ESI): m/z 498.5 (M−1)⁻.

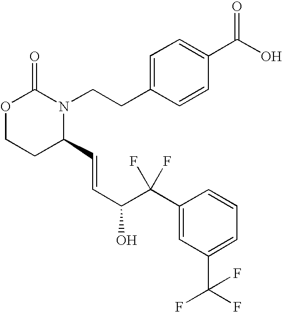

### Example 9

### Isopropyl 4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoate

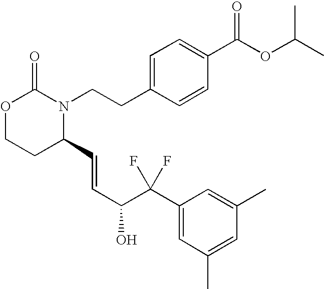

MS (+ESI): m/z 502.4 (M+1)⁺.

### Example 10

### 4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoic acid

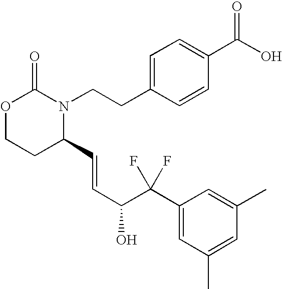

The ester from above was treated with LiOH in Methanol/water to give the corresponding acid. MS (−ESI): m/z 458.6 (M−1)⁻.

### Example 11

### 4-(2-{(4S)-4-[(3R)-4-(3,5-dimethylphenyl)-4,4-difluoro-3-hydroxybutyl]-2-oxo-1,3-oxazinan-3-yl}ethyl)benzoic acid

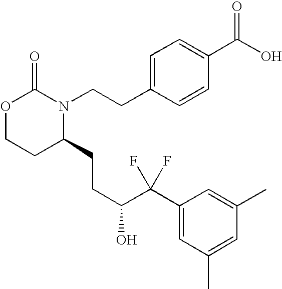

The acid from above was treated with PtO₂ in ethyl acetate/acetone under hydrogen (1 atm) to give corresponding acid. MS (+ESI): m/z 462.3 (M+1)⁺.

### Example 12

### 4-(2-{(4S)-4-[(3R)-4,4-difluoro-3-hydroxy-4-phenylbutyl]-2-oxo-1,3-oxazinan-3-yl}ethyl)benzoic acid

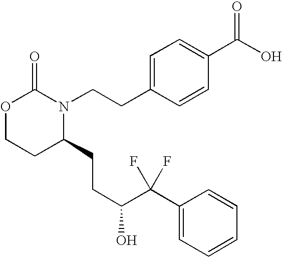

To a solution of the acid in Example 2 (37.0 mg, 0.0740 mmol) in ethanol (10 mL) was added Pd/C (5% on carbon, 5 mg). The resulting black reaction mixture was subjected to H₂ (1 atm) for 18 h. The solution was filtered over a pad of celite and the organic solvent was removed in vacuo. The crude product was purified by flash column chromatography (2% AcOH/EtOAc) to afford the title compound as a colorless oil. MS (−ESI): m/z 432.0 (M−1)⁻.

### Example 13

### Isopropyl 4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoate

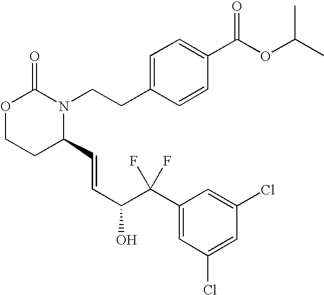

MS (+ESI): m/z 542.2 (M+H)⁺.

### Example 14

### 4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoic acid

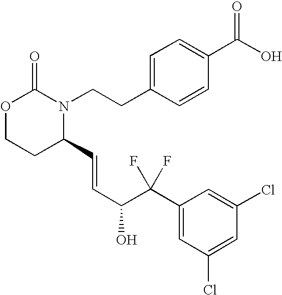

The ester from above was treated with LiOH in Methanol/water to give the corresponding acid. MS (−ESI): m/z 498.5 (M−H)⁻.

### Example 15

### 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)cyclohexanecarboxylic acid

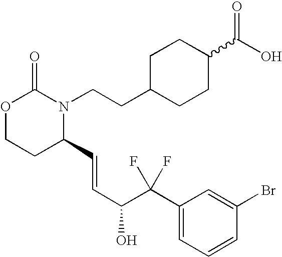

¹H NMR δ(ppm)(Acetone-d₆): a mixture of cis/trans isomer. 7.71 (2H, s), 7.57 (1H, d, J=7.4 Hz), 7.46 (1H, t, J=8.0 Hz), 5.90-5.82 (1H, m), 5.76-5.68 (1H, m), 5.26 (1H, s), 4.73 (1H, s), 4.11 (3H, m), 3.64-3.54 (1H, m), 2.83-2.53 (1H, m), 2.26-2.12 (1H, m), 1.98 (2H, m), 1.82 (2H, m), 1.59-1.19 (8H, m), 1.06-0.92 (1H, m). MS (−ESI): 514.5, 516.5.

### Example 16

### 4-(2-{(4S)-4-[(3R)-4-(3-bromophenyl)-4,4-difluoro-3-hydroxybutyl]-2-oxo-1,3-oxazinan-3-yl}ethyl)cyclohexanecarboxylic acid

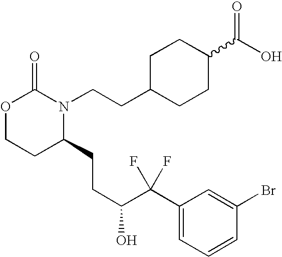

MS (−ESI): 516.3, 518.3.

The advanced intermediate 17 (WO 2004/085430) was converted to the following examples according to Scheme 6.

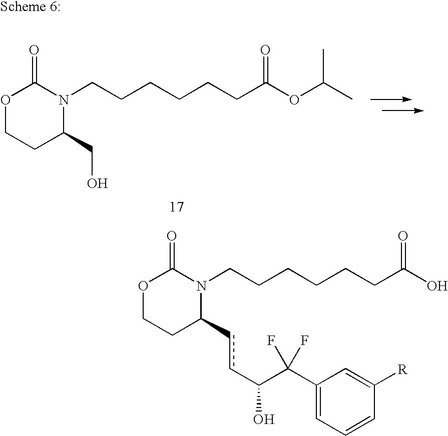

### Example 17

### 7-{(4R)-4-[(1E,3R)-4-(3-bromophenyl)-4,4-difluoro-3-hydroxybut-1-en-1-yl]-2-oxo-1,3-oxazinan-3-yl}heptanoic acid

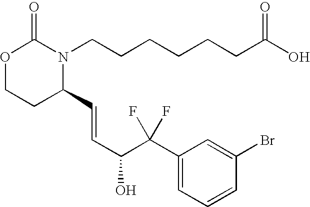

¹H NMR δ (ppm)(Acetone-d₆): 7.69 (2H, t, J=7.3 Hz), 7.55 (1H, t, J=9.4 Hz), 7.46 (1H, t, J=8.1 Hz), 5.89-5.79 (1H, m), 5.76-5.68 (1H, m), 5.32-4.96 (1H, m), 4.76-4.70 (1H, m), 4.14-4.08 (3H, m), 3.62-3.48 (1H, m), 2.74 (1H, m), 2.30 (2H, t, J=7.4 Hz), 2.21-2.11 (1H, m), 1.84-1.76 (1H, m), 1.65-1.55 (4H, m), 1.40-1.30 (4H, m).

### Example 18

### 7-{(4S)-4-[4-(3-bromophenyl)-4,4-difluoro-3-hydroxybutyl]-2-oxo-1,3-oxazinan-3-yl}heptanoic acid

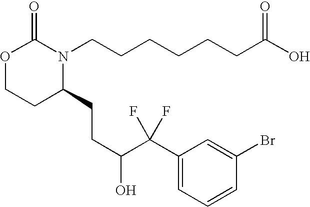

¹H NMR δ (ppm)(Acetone-d₆): 7.92 (2H, d, J=12.2 Hz), 7.79 (1H, d, J=7.6 Hz), 7.68 (1H, t, J=7.8 Hz), 5.14 (1H, d, J=15.8 Hz), 4.51-4.44 (1H, m), 4.35-4.27 (2H, m), 3.73 (2H, s), 3.27-3.19 (1−APCI): m/z 490.0, 492.0H, m), 2.51 (2H, t, J=7.3 Hz), 2.31-2.25 (4H, m), 2.16-1.52 (10H, m). MS (−APCI): m/z 490.0, 492.0.

### Example 19

### 7-{(4R)-4-[(1E,3R)-4-biphenyl-3-yl-4,4-difluoro-3-hydroxybut-1-en-1-yl]-2-oxo-1,3-oxazinan-3-yl}heptanoic acid

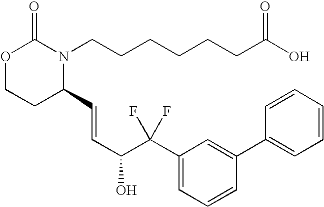

¹H NMR δ (ppm)(Acetone-d₆): 10.51 (1H, s), 7.80 (2H, d, J=10.1 Hz), 7.71 (2H, d, J=8.0 Hz), 7.60-7.50 (4H, m), 7.42 (1H, t, J=7.3 Hz), 5.88-5.80 (1H, m), 5.78-5.70 (1H, m), 5.16 (1H, s), 4.77 (1H, m), 4.12-4.06 (3H, m), 3.53-3.45 (1H, m), 2.69 (1H, dd, J=0.0, 6.7 Hz), 2.31-2.27 (2H, m), 2.16-2.10 (1H, m), 1.99 (1H, s), 1.80-1.76 (1H, m), 1.60-1.45 (4H, m), 1.35-1.29 (2H, m), 1.25-1.19 (2H, m). MS (−APCI): m/z 486.1 (M−1)⁻.

### Example 20

### 7-{(4R)-4-[(1E,3R)-4,4-difluoro-3-hydroxy-4-(2′-methylbiphenyl-3-yl)but-1-en-1-yl]-2-oxo-1,3-oxazinan-3-yl}heptanoic acid

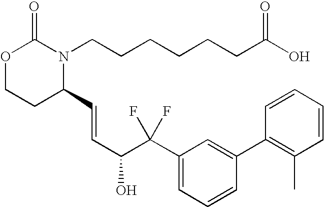

¹H NMR δ (ppm)(Acetone-d₆): 7.57 (2H, t, J=6.8 Hz), 7.50 (2H, d, J=5.1 Hz), 7.34-7.24 (4H, m), 5.86 (1H, dd, J=6.4, 15.5 Hz), 5.79-5.69 (1H, m), 5.15 (1H, s), 4.76-4.70 (1H, m), 4.12-4.04 (3H, m), 3.53-3.45 (1H, m), 2.76-2.66 (1H, m), 2.30-2.26 (5H, m), 2.18-2.10 (1H, m), 1.81-1.75 (1H, m), 1.62-1.46 (4H, m), 1.37-1.19 (5H, m). MS (−APCI): m/z 500.2 (M−1)⁻.

Other Examples in the current invention can be further prepared according to Scheme 7.

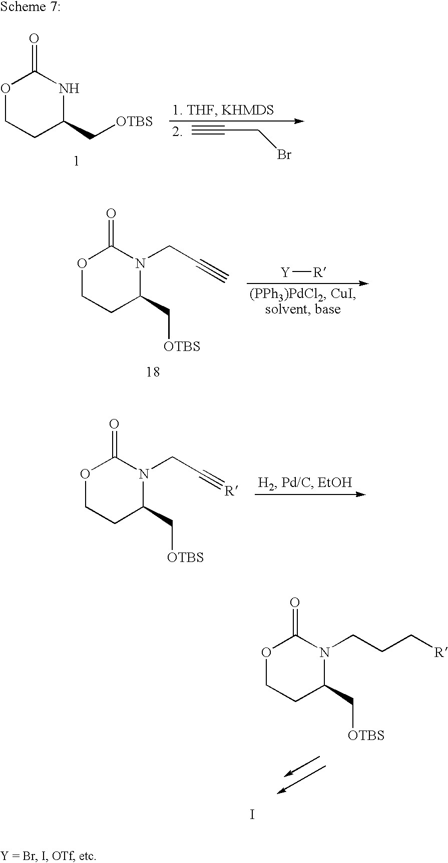

### Example 21

### methyl 4-(3-{(4R)-4-[(1E,3R)-4-(3-bromophenyl)-4,4-difluoro-3-hydroxybut-1-en-1-yl]-2-oxo-1,3-oxazinan-3-yl}propyl)benzoate

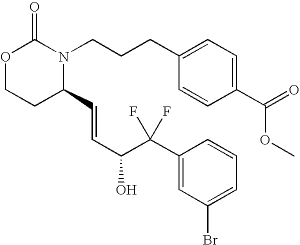

### Step 1: (4R)-4-({[tert-butyl(dimethyl)silyl]oxy}methyl)-3-prop-2-yn-1-yl-1,3-oxazinan-2-one (18)

To a solution of oxazinane 1 (1.00 g, 4.11 mmol) in THY (80 mL) at 0° C. was added KHMDS (0.5 M in toluene, 9.0 mL, 4.5 mmol). The solution was stirred for 0.5 h. Propargyl bromide (80% wt in toluene, 1.10 mL, 10.3 mmol) was added dropwise and the solution was stirred for 2 h. The resulting brown solution was heated at 50° C. for 1.5 h. The reaction was monitored by MS. The reaction was quenched with saturated NH₄Cl (aq) and the aqueous layer was extracted with EtOAc (3×). The combined organic layer was dried over Na₂SO₄, concentrated in vacuo and the crude product was purified by flash column chromatography (60% EtOAc/hexanes) to afford the desired compound as a light brown oil. MS (+ESI) m/z 284.2 (M+1)⁺.

### Step 2: Methyl 4-{3-[(4R)-4-({[tert-butyl(dimethyl)silyl]oxy}methyl)-2-oxo-1,3-oxazinan-3-yl]prop-1-yn-1-yl}benzoate

To a solution of (PPh₃)₂PdCl₂ (12.3 mg, 0.018 mmol) and CuI (3.3 mg, 0.018 mmol) in Et₂NH (2 mL) under nitrogen was cannulated the alkyne from above (99.0 mg, 0.349 mmol) in Et₂NH (2 mL). Methyl 4-iodobenzoate (91.5 mg, 0.349 mmol) was added and the reaction was stirred for 18 h at room temperature. The reaction was saturated NH₄Cl (aq) and the aqueous layer was extracted with CH₂Cl₂ (3×). The combined organic layer was dried over Na₂SO₄, filtered and concentrated in vacuo. The crude product was purified by flash column chromatography (40% EtOAc/hexanes) to afford the desired product as a dark yellow solid. MS (+ESI) m/z 418.4 (M+1)⁺.

### Step 3: Methyl 4-{3-[(4R)-4-({[tert-butyl(dimethyl)silyl]oxy}methyl)-2-oxo-1,3-oxazinan-3-yl]propyl}benzoate

To a solution of Methyl 4-{3-[(4R)-4-({[tert-butyl(dimethyl)silyl]oxy}methyl)-2-oxo-1,3-oxazinan-3-yl]prop-1-yn-1-yl}benzoate (1.13 g, 2.71 mmol) in EtOH (30 mL) was added Pd/C (10% Pd) (408 mg). The mixture was vigorously shaken under H₂ (40 psi) for 18 h. The reaction mixture was filtered over a pad of celite and concentrated in vacuo. The crude product was purified by flash column chromatography (40% EtOAc/hexanes) to afford ester Methyl 4-{3-[(4R)-4-({[tert-butyl(dimethyl)silyl]oxy}methyl)-2-oxo-1,3-oxazinan-3-yl]propyl}benzoate (1.2 g) as a brown oil.

### Example 22

### The Ester from Above was Processed to the Title Compound as Depicted in Scheme 3. MS (ESI): m/z 538.3, 540.3.

### Example 23

### 4-(3-{(4R)-4-[(1E,3R)-4-(3-bromophenyl)-4,4-difluoro-3-hydroxybut-1-en-1-yl]-2-oxo-1,3-oxazinan-3-yl}propyl)benzoic acid

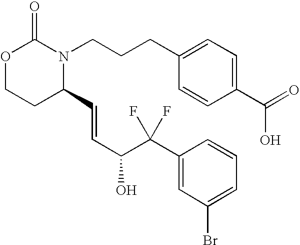

Title compound was prepared according to hydrolysis described in Scheme 3. MS (+ESI): m/z 524.1, 526.1.

### Example 24

### methyl 6-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)pyridine-2-carboxylate

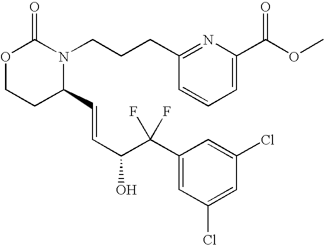

Title compound was prepared according to Schemes 7 and 3. In the palladium-catalyzed coupling reaction, THF was used as the solvent and Et₃N (2.4 eq) as the base.

MS (+ESI): m/z 529.2 (M+1)⁺.

### Example 25

### 6-(3-{(4R)-4-[(1E,3R)-4-(3,5-dichlorophenyl)-4,4-difluoro-3-hydroxybut-1-en-1-yl]-2-oxo-1,3-oxazinan-3-yl}propyl)pyridine-2-carboxylic acid

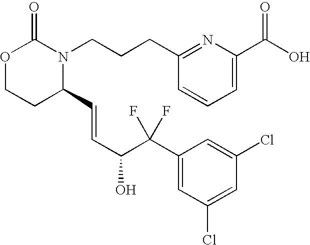

Title compound was prepared according to hydrolysis described in Scheme 3.

MS (+ESI): m/z 515.1 (M+1)⁺, 517.1 (M+2)⁺.

### Example 26

### Methyl 2-(3-{(4R)-4-[(1E,3R)-4-(3-bromophenyl)-4,4-difluoro-3-hydroxybut-1-en-1-yl]-2-oxo-1,3-oxazinan-3-yl}propyl)-1,3-thiazole-5-carboxylate

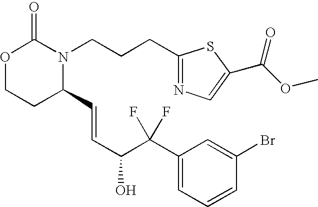

Title compound was prepared according to Schemes 7 and 3. In the palladium-catalyzed coupling reaction, THE was used as solvent with Et₃N (2.4 eq) as base.

MS (+ESI): m/z 544.9, 547.1.

### Example 27

### 2-(3-{(4R)-4-[(1E,3R)-4-(3-bromophenyl)-4,4-difluoro-3-hydroxybut-1-en-1-yl]-2-oxo-1,3-oxazinan-3-yl}propyl)-1,3-thiazole-5-carboxylic acid

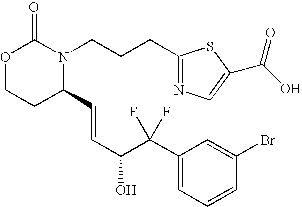

Title compound was obtained from hydrolysis of the methyl ester in Example 26 by LiOH.

MS (+ESI): m/z 531.0, 533.0.

### Example 28

### 3-(3-{(4R)-4-[(1E,3R)-4-(3-bromophenyl)-4,4-difluoro-3-hydroxybut-1-en-1-yl]-2-oxo-1,3-oxazinan-3-yl}propyl)benzoic acid

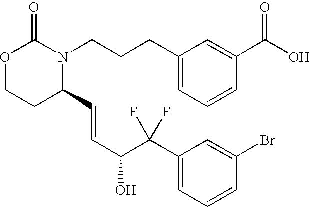

MS (+ESI): m/z 526.0, 528.0.

### Example 29

### 3-(3-{(4S)-4-[(3R)-4-(3-bromophenyl)-4,4-difluoro-3-hydroxybutyl]-2-oxo-1,3-oxazinan-3-yl}propyl)benzoic acid

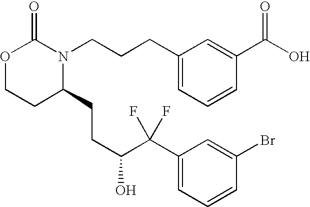

MS (+ESI): m/z 528.0, 530.0.

### Example 30

### 5-(3-{(4R)-4-[(1E,3R)-4-(3-bromophenyl)-4,4-difluoro-3-hydroxybut-1-en-1-yl]-2-oxo-1,3-oxazinan-3-yl}propyl)isoxazole-3-carboxylic acid

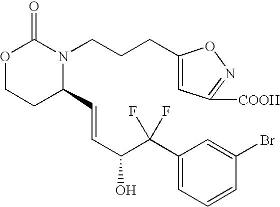

### I. Effects of an EP4 Agonist on Intraocular Pressure (IOP) in Rabbits and Monkeys.

### Animals

Drug-näive, male Dutch Belted rabbits and female cynomolgus monkeys are used in this study. Animal care and treatment in this investigation are in compliance with guidelines by the National Institute of Health (NIH) and the Association for Research in Vision and Opthalmology (ARVO) resolution in the use of animals for research. All experimental procedures str approved by the Institutional Animal Care and Use Committee of Merck and Company.

### Drug Preparation and Administration

Drug concentrations are expressed in terms of the active ingredient (base). The compounds of this invention are dissolved in a suitable ophthalmic solution (e.g., 0.5% Polysorbate-80, 0.02% benzalkonium chloride, 0.1% EDTA, 4.5% mannitol in 5 mM citrate) at 22, 2.2 and 0.22 μM for rabbit study and 111, 33, 11, and 1.1 μM for monkey studies. Drug or vehicle aliquots (25 ul) are administered topically unilaterally or bilaterally. In unilateral applications, the contralateral eyes receive an equal volume of vehicle. Proparacaine (0.5%) is applied to the cornea prior to tonometry to minimize discomfort. Intraocular pressure (IOP) is recorded using a pneumatic tonometer (Alcon Applanation Pneumatonograph) or equivalent.

### Analysis

The results are expressed as the changes in IOP from the basal level measured just prior to administration of drug or vehicle and represent the mean, plus or minus standard deviation. Statistical comparisons are made using the Student's t-test for non-paired data between responses of drug-treated and vehicle-treated animals and for paired data between ipsilateral and contralateral eyes at comparable time intervals. The significance of the date is also determined as the difference from the “t-0” value using Dunnett's “t” test. Asterisks represent a significance level of p<0.05.

### A. Intraocular Pressure Measurement in Rabbits

Male Dutch Belted rabbits weighing 2.5-4.0 kg are maintained on a 12-hour light/dark cycle and rabbit chow. All experiments are performed at the same time of day to minimize variability related to diurnal rhythm. IOP is measured before treatment then the compounds of this invention or vehicle are instilled (one drop of 25 ul) into one or both eyes and IOP is measured at 30, 60, 120, 180, 240, 300, and 360 minutes after instillation. In some cases, equal number of animals treated bilaterally with vehicle only are evaluated and compared to drug treated animals as parallel controls.

### B. Intraocular Pressure Measurements in Monkeys.

Unilateral ocular hypertension of the right eye is induced in female cynomolgus monkeys weighing between 2 and 3 kg by photocoagulation of the trabecular meshwork with an argon laser system (Coherent NOVUS 2000, Palo Alto, USA) using the method of Lee at al. (1985). The prolonged increase in intraocular pressure (IOP) results in changes to the optic nerve head that are similar to those found in glaucoma patients.

For IOP measurements, the monkeys are kept in a sitting position in restraint chairs for the duration of the experiment. Animals are lightly anesthetized by the intramuscular injection of ketamine hydrochloride (3-5 mg/kg) approximately five minutes before each IOP measurement and one drop of 0.5% proparacaine was instilled prior to recording IOP. IOP is measured using a pneumatic tonometer (Alcon Applanation Tonometer) or a Digilab pneumatonometer (Bio-Rad Ophthalmic Division, Cambridge, Mass., USA).

IOP is measured before treatment and generally at 30, 60, 124, 180, 300, and 360 minutes after treatment. Baseline values are also obtained at these time points generally two or three days prior to treatment. Treatment consists of instilling one drop of 25 ul of the compounds of this invention (1.1 to 111 μM) or vehicle (0.5% Polysorbate-80, 0.02% benzalkonium chloride, 0.1% EDTA, 4.5% mannitol in 5 mM citrate). At least one-week washout period is employed before testing on the same animal. The normotensive (contralateral to the hypertensive) eye is treated in an exactly similar manner to the hypertensive eye. IOP measurements for both eyes are compared to the corresponding baseline values at the same time point. Results are expressed as mean plus-or-minus standard deviation in mm Hg. The activity range of the compounds of this invention for ocular use is between 0.01 and 100,000 nM.

Compounds from the current invention (i.e., Example 1) showed improved ocular tolerability in animal species such as rabbits and cynomolgus monkeys compared to compounds disclosed in WO 2004/085430 (i.e., Example 2). For example, in a vehicle panel-controlled study in New Zealand white rabbits, a single dose (topical, unilateral) of 2.2 μM (25 μL) of an ophthalmic solution of Example 3 or vehicle (0.5% Polysorbate-80, 0.02% benzalkonium chloride, 0.1% EDTA, 4.5% mannitol in 5 mM citrate) induced very slight drug treatment related ocular adverse effects (eye closure). Under the same treatment paradigm, Example 2 in WO 2004/085430 caused more profound eye closure.

### II. Radioligand Binding Assays:

The assays used to test these compounds were performed essentially as described in: Abramovitz M, Adam M, Boie Y, Carriere M, Denis D, Godbout C, Lamontagne S, Rochette C, Sawyer N, Tremblay N M, Belley M, Gallant M, Dufresne C, Gareau Y, Ruel R, Juteau H, Labelle M, Ouimet N, Metters K M. The utilization of recombinant prostanoid receptors to determine the affinities and selectivities of prostaglandins and related analogs. Biochim Biophys Acta 2000 Jan. 17; 1483(2):285-293 and discussed below:

### Stable Expression of Prostanoid Receptors in the Human Embryonic Kidney (HEK) 293(EBNA) Cell Line

Prostanoid receptor (PG) cDNAs corresponding to full length coding sequences were subcloned into the appropriate sites of the mammalian expression vector pCEP4 (Invitrogen) pCEP4PG plasmid DNA was prepared using the Qiagen plasmid preparation kit (QIAGEN) and transfected into HEK 293(EBNA) cells using LipofectAMINE@(GIBCO-BRL) according to the manufacturers' instructions. HEK 293(EBNA) cells expressing the cDNA together with the hygromycin resistance gene were selected in Dulbecco's Modified Eagle Medium (DMEM) supplemented with 10% heat inactivated fetal bovine serum, 1 mM sodium pyruvate, 100 U/ml Penicillin-G, 100 μg/ml Streptomycin sulphate, 250 μg/ml active GENETICIN™(G418) (all from Life Technologies, Inc./BRL) and 200 μg/ml hygromycin (Calbiochem). Individual colonies were isolated after 2-3 weeks of growth under selection using the cloning ring method and subsequently expanded into clonal cell lines. Expression of the receptor cDNA was assessed by receptor binding assays.

HEK 293(EBNA) cells were grown in supplemented DMEM complete medium at 37° C. in a humidified atmosphere of 6% CO₂ in air, then harvested and membranes prepared by differential centrifugation (1000×g for 10 min, then 160,000×g for 30 min, all at 4° C.) following lysis of the cells by nitrogen cavitation at 800 psi for 30 min on ice in the presence of protease inhibitors (2 mM phenylmethylsulfonylfluoride, 10 μM E-64, 100 μM leupeptin and 0.05 mg/ml pepstatin). The 160,000×g pellets were resuspended in 10 mM HEPES/KOH (pH 7.4) containing 1 mM EDTA at approximately 5-10 mg/ml protein by Dounce homogenisation (Dounce A; 10 strokes), frozen in liquid nitrogen and stored at −80° C.

### Prostanoid Receptor Binding Assays

Prostanoid receptor binding assays were performed in a final incubation volume of 0.2 mL in 10 mM MES/KOH (pH 6.0) (EP subtypes, FP and TP) or 10 mM HEPES/KOH (pH 7.4) (DP and IP), containing 1 mM EDTA, 10 mM MgCl₂ (EP subtypes) or 10 mM MnCl₂ (DP, FP, IP and TP) and radioligand [0.5-1.0 nM [³H]PGE₂ (181 Ci/mmol) for EP subtypes, 0.7 nM [³H]PGD₂ (115 Ci/mmol) for DP, 0.95 nM [³H]PGF_{2α}, (170 Ci/mmol) for FP, 5 nM [³H]iloprost (16 Ci/mmol) for IP and 1.8 nM [³H]SQ 29548 (46 Ci/mmol) for TP]. EP₃ assays also contained 100 μM GTPγS. The reaction was initiated by addition of membrane protein (approximately 30 μg for EP₁, 20 μg for EP₂, 2 μg for EP₃, 10 μg for EP₄, 60 μg for FP, 30 μg for DP, 10 μg for IP and 10 μg for TP) from the 160,000×g fraction. Ligands were added in dimethylsulfoxide (Me₂SO) which was kept constant at 1% (v/v) in all incubations. Non-specific binding was determined in the presence of 1 μM of the corresponding non-radioactive prostanoid. Incubations were conducted for 60 min (EP subtypes, FP and IP) or 30 min (DP and TP) at 30° C. (EP subtypes, DP, FP and TP) or room temperature (IP) and terminated by rapid filtration through a 96-well Unifilter GF/C (Canberra Packard) prewetted in assay incubation buffer without EDTA (at 4° C.) and using a Tomtec Mach III 96-well semi-automated cell harvester. The filters were washed with 3-4 mL of the same buffer, dried for 90 min at 55° C. and the residual radioactivity bound to the individual filters determined by scintillation counting with addition of 50 μl of Ultima Gold F (Canberra Packard) using a 1450 MicroBeta (Wallac). Specific binding was calculated by subtracting non-specific binding from total binding. Specific binding represented 90-95% of the total binding and was linear with respect to the concentrations of radioligand and protein used. Total binding represented 5-10% of the radioligand added to the incubation media.

The activity range of the compounds of this invention for bone use is between 0.01 and 100,000 nM.

### Bone Resorption Assays:

### 1. Animal Procedures:

For mRNA localization experiments, 5-week old Sprague-Dawley rats (Charles River) are euthanized by CO₂, their tibiae and calvariae are excised, cleaned of soft tissues and frozen immediately in liquid nitrogen. For EP₄ regulation experiments, 6-week old rats are given a single injection of either vehicle (7% ethanol in sterile water) or an anabolic dose of PGE₂ (Cayman Chemical, Ann Arbor, Mich.), 3-6 mg/kg in the same vehicle) intraperitoneally. Animals are euthanized at several time points post-injection and their tibiae and calvariae, as well as samples from lung and kidney tissues are frozen in liquid nitrogen.

### 2. Cell Cultures

RP-1 periosteal cells are spontaneously immortalized from primary cultures of periosteal cells from tibae of 4-week old Sprague-Dawley rats and are cultured in DMEM (BRL, Gaithersburg, Md.) with 10% fetal bovine serum (JRH Biosciences, Lenexa, Kans.). These cells do not express osteoblastic phenotypic markers in early culture, but upon confluence, express type I collagen, alkaline phosphatase and osteocalcin and produce mineralized extracellular matrix.

RCT-1 and RCT-3 are clonal cell lines immortalized by SV-40 large T antigen from cells released from fetal rat calvair by a combination collagenase/hyaluronidase digestion. RCT-1 cells, derived from cells released during the first 10 minutes of digestion (fraction I), are cultured in RPMI 1640 medium (BRL) with 10% fetal bovine serum and 0.4 mg/ml G418 (BRL). These cells differentiate and express osteoblastic features upon retinoic acid treatment. RCT-3 cells, immortalized from osteoblast-enriched fraction III cells, are cultured in F-12 medium (BRL) with 5% Fetal bovine serum and 0.4 mg/ml G418. TRAB-11 cells are also immortalized by SV40 large T antigen from adult rat tibia and are cultured in RPMI 1640 medium with 10% FBS and 0.4 mg/ml G418. ROS 17/2.8 rat osteosarcoma cells are cultured in F-12 containing 5% FBS. Osteoblast-enriched (fraction III) primary fetal rat calvaria cells are obtained by collagenase/hyaluronidase digestion of calvariae of 19 day-old rat fetuses. See Rodan et al., Growth stimulation of rat calvaria osteoblastic cells by acidic FGF, Endocrinology, 121, 1919-1923 (1987), which is incorporated by reference herein in its entirety. Cells are released during 30-50 minutes digestion (fraction 1H) and are cultured in F-12 medium containing 5% FBS.

P815 (mouse mastocytoma) cells, cultured in Eagles MEM with 10% FBS, and NRK (normal rat kidney fibroblasts) cells, cultured in DMEM with 10% FBS, are used as positive and negative controls for the expression of EP₄, respectively. See Abramovitz et al., Human prostanoid receptors: cloning and characterization. In: Samulesson B. et al. ed) Advances in prostaglandin, Thrombosznes and leukotriene research, vol. 23, pp. 499-504 (1995) and de Larco et al., Epithelioid and fibroblastic rat kidney cell clones. EGF receptors and the effect of mouse sarcoma virus transformation, Cell Physiol., 94, 335-342 (1978), which are both incorporated by reference herein in their entirety.

### 3. Northern Blot Analysis:

Total RNA is extracted from the tibial metaphysis or diaphysis and calvaria using a guanidinium isothiocyanate-phenol-chloroform method after pulverizing frozen bone samples by a tissue homogenizer. See P. Chomczynski et al., Single-step method of RNA isolation by acid guanidium thiocyanate-phenol-chloroform extraction., Analyt Biochem, 162, 156-159 (1987), which is incorporated by reference herein in its entirety. RNA samples (20 mg) are separated on 0.9% agarose/formaldehyde gels and transferred onto nylon membranes (Boehringer Mannheim, Germany). Membranes are prehybridized in Hybrisol I (Oncor, Gaithersburg, Md.) and 0.5 mg/ml sonicated salmon sperm DNA (Boehringer) at 42° C. for 3 hours and are hybridized at 42° C. with rat EP₂ and mouse EP₄ cDNA probes labeled with [³²P]-dCTP (Amersham, Buckinghamshire, UK) by random priming using the rediprime kit (Amersham). After hybridization, membranes are washed 4 times in 2×SSC+0.1% SDS at room temperature for a total of 1 hour and once with 0.2×SSC+0.1% SDS at 55° C. for 1 hour and then exposed to Kodak XAR 2 film at −70° C. using intensifying screens. After developing the films, bound probes are removed twice with 0.1% SDS at 80° C. and membranes are hybridized with a human GAPDH (Glyceraldehyde 3-Phosphate Dehydrogenase) cDNA probe (purchased from Clontech, Palo Alto, Calif.) for loading control.

### 4. In-Situ Hybridization:

Frozen tibiae are sectioned coronally at 7 mm thickness and sections are mounted on charged slides (Probe On Plus, Fisher Scientific, Springfield, N.J.) and are kept at −70° C. until hybridization. cRNA probes are labeled with ³⁵S-UTPgS (ICN, Costa Mesa, Calif.) using a Riboprobe II kit (Promega Madison, Wis.). Hybridization is performed overnight at 50° C. See M Weinreb et al., Different pattern of alkaline phosphatase, osteopontin and osteocalcin expression in developing rat bone visualized by in-situ hybridization, J Bone Miner Res., 5, 831-842 (1990) and D. Shinar et al., Expression of alphav and beta3 integrin subunits in rat osteoclasts in situ, J. Bone Miner. Res., 8, 403-414 (1993), which are both incorporated by reference herein in their entirety. Following hybridization and washing, sections are dipped in Ilford K5 emulsion diluted 2:1 with 6% glycerol in water at 42° C. and exposed in darkness at 4° C. for 12-14 days. Slides are developed in Kodak D-19 diluted 1:1 with water at 15°, fixed, washed in distilled water and mounted with glycerol-gelatin (Sigma) after hematoxylin staining. Stained sections are viewed under the microscope (Olympus, Hamburg, Germany), using either bright-field or dark-field optics.

### 5. Expression of EP₄ in Osteoblastic Cell Lines and in Bone Tissue.

The expression of EP₄ and EP₂ mRNA is examined in various bone derived cells including osteoblast-enriched primary rat calvaria cells, immortalized osteoblastic cell lines from fetal rat calvaria or from adult rat tibia and an osteoblastic osteosarcoma cell line. Most of the osteoblastic cells and cell lines show significant amounts of 3.8 kb EP₄ mRNA, except for the rat osteosarcoma cell line ROS 17/2.8. Consistent with this finding, in ROS 17/2.8 cells PGE₂ has no effect on intracellular cAMP, which is markedly induced in RCT-3 and TRAB-11 cells. Treatment of RCT-1 cells with retinoic acid, which promotes their differentiation, reduces the levels of EP₄ mRNA. NRK fibroblasts do not express EP₄ mRNA, while P815 mastocytoma cells, used as positive controls, express large amounts of EP₄ mRNA. In contrast to EP₄ mRNA, none of the osteoblastic cells and cell lines express detectable amounts of EP₂ mRA in total RNA samples. Expression of EP₄ mRNA in osteoblastic cells, EP₄ is also expressed in total RNA isolated from tibiae and calvariae of 5-week-old rats. In contrast, no EP₂ mRNA is found in RNA from tibial shafts.

6. PGE₂ Induces the Expression of EP₄ mRNA in RP-1 Periosteal Cells and in Adult Rat Tibiae

PGE₂ enhances its own production via upregulation of cyclooxygenase 2 expression in osteoblasts and in bone tissue thus autoamplifying its own effects. PGE₂ also increases the levels of EP₄ mRNA. RP-1 cells are immortalized from a primary culture of adult rat tibia periosteum is examined. These cells express osteoblast phenotypic markers upon confluence and form mineralized bone matrix when implanted in nude mice. Similar to the other osteoblastic cells examined, RP-1 periosteal cells express a 3.8 kb EP₄ transcript. Treatment with PGE₂ (10⁻⁶ M) rapidly increases EP₄ mRNA levels peaking at 2 hours after treatment. PGE₂ has no effect on EP₄ mRNA levels in the more differentiated RCT-3 cells pointing to cell-type specific regulation of EP₄ expression by PGE₂. EP₂ mRNA is not expressed in RP-1 cells before or after treatment with PGE₂.

To examine if PGE₂ regulates EP₄ mRNA levels in vivo in bone tissue, five-week-old male rats are injected with PGE₂ (3-6 mg/Kg). Systemic administration of PGE₂ rapidly increased EP₄ mRNA levels in the tibial diaphysis peaking at 2 h after injection. A similar effect of PGE₂ on EP₄ mRNA is observed in the tibial metaphysis and in calvaria. PGE2 induces EP₄ mRNA levels in vitro in osteogenic periosteal cells and in vivo in bone tissue in a cell type-specific and tissue-specific manner. PGE₂ does not induce EP₂ mRNA in RP-1 cells nor in bone tissue.

7. Localization of EP₄ mRNA Expression in Bone Tissue

In situ hybridization is used in order to localize cells expressing EP₄ in bone. In control experiment (vehicle-injected) rats, low expression of EP₄ is detected in bone marrow cells. Administration of a single anabolic dose of PGE₂ increased the expression of EP₄ in bone marrow cells. The distribution of silver grains over the bone marrow is not uniform and occurs in clumps or patches in many areas of the metaphysis. Within the tibial metaphysis, EP₄ expression is restricted to the secondary spongiosa area and is not seen in the primary spongiosa. Hybridization of similar sections with a sense probe (negative control) does not show any signal.

EP₄ is expressed in osteoblastic cells in vitro and in bone marrow cells in vivo, and is upregulated by its ligand, PGE₂.

### 8. Agonists of the Present Invention

Using standard methods for measuring agonist activity, the following compounds are evaluated in cell cultures and in EP₄ receptor cell-free systems to determine the agonist activity of the compounds in terms of their EC₅₀ value.

What is claimed is:

1. A compound having the structural formula I: 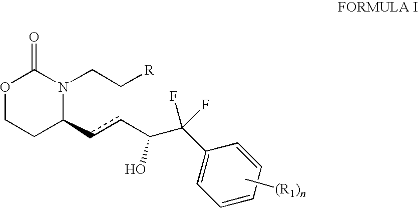
or a pharmaceutically acceptable salt, enantiomer, diastereomer, prodrug or mixture thereof, wherein,
R represents (CH₂)ₓCOOR₃, (CH₂)ₙC₃₋₁₀ cycloalkyl; —(CH₂)ₙC₃₋₁₀ heterocyclyl, (CH₂)ₙC₆₋₁₀ aryl, said cycloalkyl , heterocyclyl, and aryl substituted with R₂; provided that when R is —(CH₂)ₙC₃₋₁₀ heterocyclyl it does not represent thienyl;
R₁ independently represents C₁₋₆ alkyl, halogen, CF₃, or aryl, said aryl optionally substituted with 1 to 3 groups of halogen, C₁₋₆ alkyl, CF₃, or N(R₄)₂;
R₂ represents COOR₃ or a carboxylic acid isostere;
R₃ and R₄ independently represent H, or C₁₋₆ alkyl;
n represents 1-3;
x represents 2-5; and
--- represents a double or single bond.

2. A compound according to claim 1 wherein R is (CH₂)ₓCOOR₃.

3. A compound according to claim 1 wherein R is (CH₂)ₙC₆₋₁₀ aryl, which is

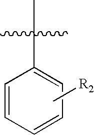

4. A compound according to claim 3 wherein R₁ is halogen, C₁₋₆ alkyl or CF₃.

5. A compound according to claim 1 wherein R is (CH₂)ₙC₆₋₁₀ aryl, which is 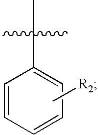
R₂ is COOH, COOCH(CH₃)₂, or tetrazole, and, R₁ is halogen.

6. A compound according to claim 1 wherein R is (CH₂)ₓCOOR₃, x is 3-4, R₁ is halogen and R₃ is COOH.

7. A compound which is:
Isopropyl 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoic acid;
Isopropyl 4-(2-{(4S)-4-[(3R)-4-(3 bromophenyl)-4,4-difluoro-3-hydroxybutyl]-2-oxo-1,3-oxazinan-3-yl}ethyl)benzoate;
4-(2-{(4S)-4-[(3R)-4-(3-bromophenyl)-4,4-difluoro-3-hydroxybutyl]-2-oxo-1,3-oxazinan-3-yl}ethyl)benzoic acid;
Isopropyl 4-[2-((4S)-4-{(3R)-4,4-difluoro-3-hydroxy-4-[3-(trifluoromethyl)phenyl]butyl}-2-oxo-1,3-oxazinan-3-yl)ethyl]benzoate;
4-[2-((4S)-4-{(3R)-4,4-difluoro-3-hydroxy-4-[3-(trifluoromethyl)phenyl]butyl}-2-oxo-1,3-oxazinan-3-yl)ethyl]benzoic acid;
Isopropyl 4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoate;
4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoic acid;
Isopropyl 4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoic acid;
4-(2-{(4S)-4-[(3R)-4-(3,5-dimethylphenyl)-4,4-difluoro-3-hydroxybutyl]-2-oxo-1,3-oxazinan-3-yl}ethyl)benzoic acid;
4-(2-{(4S)-4-[(3R)-4,4-difluoro-3-hydroxy-4-phenylbutyl]-2-oxo-1,3-oxazinan-3-yl}ethyl)benzoic acid;
Isopropyl 4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoate;
Isopropyl 4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3-bromophenyl)-4,4-difluoro-3-hydroxybut-1-en-1-yl]-2-oxo-1,3-oxazinan-3-yl}ethyl)benzoic acid;
Isopropyl 4-(2-{(4S)-4-[(3R)-4-(3-bromophenyl)-4,4-difluoro-3-hydroxybutyl]-2-oxo-1,3-oxazinan-3-yl}ethyl)benzoate;
4-(2-{(4S)-4-[(3R)-4-(3-bromophenyl)-4,4-difluoro-3-hydroxybutyl]-2-oxo-1,3-oxazinan-3-yl}ethyl)benzoic acid;
Isopropyl 4-[2-((4S)-4-{(3R)-4,4-difluoro-3-hydroxy-4-[3-(trifluoromethyl)phenyl]butyl}-2-oxo-1,3-oxazinan-3-yl)ethyl]benzoate;
4-[2-((4S)-4-{(3R)-4,4-difluoro-3-hydroxy-4-[3-(trifluoromethyl)phenyl]butyl}-2-oxo-1,3-oxazinan-3-yl)ethyl]benzoic acid;
Isopropyl 4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoate;
4-[2-((4R)-4-{(1E,3R)-4,4-difluoro-3-hydroxy-4-[3-(trifluoromethyl)phenyl]but-1-en-1-yl}-2-oxo-1,3-oxazinan-3-yl)ethyl]benzoic acid;
Isopropyl 4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3,5-dimethylphenyl)-4,4-difluoro-3-hydroxybut-1-en-1-yl]-2-oxo-1,3-oxazinan-3-yl}ethyl)benzoic acid;
4-(2-{(4S)-4-[(3R)-4-(3,5-dimethylphenyl)-4,4-difluoro-3-hydroxybutyl]-2-oxo-1,3-oxazinan-3-yl}ethyl)benzoic acid;
4-(2-{(4S)-4-[(3R)-4,4-difluoro-3-hydroxy-4-phenylbutyl]-2-oxo-1,3-oxazinan-3-yl}ethyl)benzoic acid;
Isopropyl 4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoate;
4-(2-{(4R)-4-[(1E,3R)-4-(3,5-dichlorophenyl)-4,4-difluoro-3-hydroxybut-1-en-1-yl]-2-oxo-1,3-oxazinan-3-yl}ethyl)benzoic acid;
or a pharmaceutically acceptable salt, enantiomer, diastereomer, prodrug or mixture thereof.

9. A pharmaceutical composition comprising a pharmaceutically acceptable carrier and a compound of formula I, according to any one of claims 1 to 8.

10. Use of a compound of any one of claims 1 to 8 for making a medicament for treating ocular hypertension or glaucoma.

11. The composition according to claim 9 wherein one or more active ingredients belonging to the group consisting of: β-adrenergic blocking agent, parasympatho-mimetic agent, sympathomimetic agent, carbonic anhydrase inhibitor, Maxi-K channel blocker, and a prostaglandin, hypotensive lipid, neuroprotectant, and 5-HT2 receptor agonist is optionally added.

12. The composition according to claim 11 wherein the β-adrenergic blocking agent is timolol, betaxolol, levobetaxolol, carteolol, or levobunolol; the parasympathomimetic agent is pilocarpine; the sympathomimetic agent is epinephrine, brimonidine, iopidine, clonidine, or para-aminoclonidine, the carbonic anhydrase inhibitor is dorzolamide, acetazolamide, metazolamide or brinzolamide; COSOPT®, the Maxi-K is Penitrem A, paspalicine, charybdotoxin, iberiotoxin, Paxicillan, Aflitram, Verroculogen, 1-(1-isobutyl-6-methoxy-1H-indazol-3-yl)-2-methylpropan-1-one; 1-[1-(2,2-dimethylpropyl)-6-methoxy-1H-indazol-3-yl]-2-methylpropan-1-one; 1-[1-(cyclohexylmethyl)-6-methoxy-1H-indazol-3-yl]-2-methylpropan-1-one; 1-(1-hexyl-6-methoxy-1H-indazol-3-yl)-2-methylpropan-1-one; 1-[1-(2-ethylhexyl)-6-methoxy-1H-indazol-3-yl]-2-methylpropan-1-one; 1-(3-isobutyryl-6-methoxy-1H-indazol-1-yl)buan-2-one; 1-(3-isobutyryl-6-methoxy-1H-indazol-1-yl)-3,3-dimethylbutan-2-one; 1-(3-cyclopentylcarbonyl)-6-methoxy-1H-indazol-1-yl)-3,3-dimethylbutan-2-one; 1-(3,3-dimethyl-2-oxobutyl)-6-methoxy-1H-indazole-3-carboxylic acid; and 1-[3-(3-hydroxypropanoyl)-6-methoxy-1H-indazol-1-yl]-3,3-dimethylbutan-2-one, the prostaglandin is latanoprost, travaprost, unoprostone, rescula, or S1033, the hypotensive lipid is lumigan, the neuroprotectant is eliprodil, R-eliprodil or memantine; and the 5-HT2 receptor agonist is 1-(2-aminopropyl)-3-methyl-1H-imdazol-6-ol fumarate or 2-(3-chloro-6-methoxy-indazol-1-yl)-1-methyl-ethylamine.

13. Use of a compound of any one of claims 1 to 8 for treating macular edema or macular degeneration, treating dry eye, increasing retinal and optic nerve head blood velocity, increasing retinal and optic nerve oxygen tension or providing a neuroprotection.

14. The composition according to claim 9 which is a topical formulation in the form of a solution or suspension, said composition optionally containing xanthan gum or gellan gum.

15. Use of a compound of any one of claims 1 to 8 for stimulating bone formation, treating or reducing the risk of contracting a disease state or condition related to abnormal bone resorption, in a mammal in need thereof.
